(19) United States
(12) Patent Application Publication
(10) Pub. No.: US 2011/0195951 A1
(43) Pub. Date: Aug. 11, 2011

(54) DIAZAINDOLE DERIVATIVES AND THEIR USE IN THE INHIBITION OF C-JUN N-TERMINAL KINASE

(75) Inventors: Piotr Pawel Graczyk, London (GB); Gurpreet Singh Bhatia, London (GB)

(73) Assignee: EISAI R&D MANAGEMENT CO., LTD., Tokyo (JP)

(21) Appl. No.: 13/057,448

(22) PCT Filed: Jul. 28, 2009
(86) PCT No.: PCT/GB09/01859

§ 371 Date: Apr. 28, 2011

(30) Foreign Application Priority Data
Aug. 5, 2008 (GB) 0814364.6

(51) Int. Cl. A61K 31/553; C07D 471/04; A61K 31/437; C07D 413/14; C07D 487/04; A61K 31/4985; A61K 31/5377; A61K 31/5025; A61P 25/00; A61P 29/00; A61P 19/02

(52) U.S. Cl. 514/211.15; 546/119; 514/303; 540/544; 544/350; 514/249; 544/117; 514/234.2; 544/236; 514/248

(57) ABSTRACT

The invention relates to diazaindole derivatives represented by the general formula (I): where A, E, G, R¹, R², R³ and R⁴ are defined herein, or pharmaceutically acceptable salts thereof, their use in the inhibition of c-Jun N-terminal kinase (JNK) activity, their use in medicine and particularly in the treatment of neurodegenerative disorders, inflammatory diseases, autoimmune diseases and/or organ failure. The invention also provides processes for the manufacture of said diazaindole derivatives and compositions containing them.

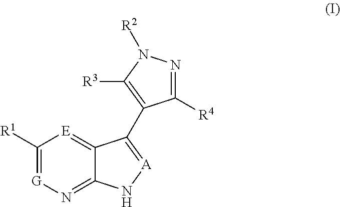

### FIELD OF THE INVENTION

The invention relates to diazaindole derivatives or pharmaceutically acceptable salts thereof, their use in the inhibition of c-Jun N-terminal kinase (JNK) activity, their use in medicine and particularly in the treatment of neurodegenerative disorders, inflammatory diseases, autoimmune diseases and/or organ failure. The invention also provides processes for the manufacture of said diazaindole derivatives and compositions containing them.

### BACKGROUND OF THE INVENTION

The c-Jun N-terminal kinases (hereinafter referred to as “JNKs”) are a family of serine/threonine protein kinases and members of the mitogen-activated protein kinase (MAPK) family. Three distinct genes (JNK1, JNK2 and JNK3) have been identified.

It is known that JNKs are related to neurodegenerative disorders such as multiple sclerosis and autoimmune diseases such as rheumatoid arthritis (WO2004/078756).

Furthermore, the above patent reference also discloses that 7-azaindole derivatives which have a ring on the C3 position and an aromatic group such as a phenyl group or a heterocyclic group such as a morpholino group on the C5 position possess JNK inhibitory activity.

Also, it is known that certain 7-azaindole derivatives having substitution (for example, a thiazolyl group) at the C3 position and substitution (for example, a heterocyclic group) at the C5 position can show in vitro inhibitory activity against other kinases, namely TEC and JAK kinases (WO2006/004984).

However, there is no disclosure of 7-azaindole derivatives which have a pyrazolyl group on the C3 position and a non-aromatic carbocyclic group on the C5 position.

Furthermore, there is no disclosure of n,7-diazaindole derivatives (where n=2, 4 or 6) which have a pyrazolyl group on the C3 position and an aromatic or non-aromatic carbocyclic group on the C5 position.

It is desirable for a JNK inhibitor to have superior selectivity for JNK over other kinases. This is in order to reduce the risk of unexpected side-effects.

### DESCRIPTION OF THE INVENTION

The present inventors have found that n,7-diazaindole derivatives which have a pyrazolyl group on C3 position and hydrocarbon cyclic group on C5 position have a superior selectivity for JNK kinases over other kinases and show significant in vitro activity, thereby completing the present invention.

The present invention provides the compound of formula (I) or a pharmaceutically acceptable salt thereof:

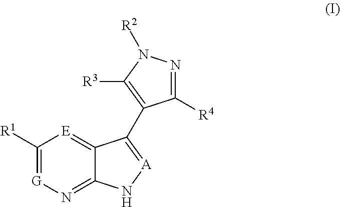

wherein

A is CH or N;

E is CH or N;

G is CH or N;

A is N when E and G are CH;

E is N when A and G are CH;

G is N when A and E are CH;

R¹ is a 5-7 membered non-aromatic hydrocarbon cyclic group optionally and independently substituted with 1-4 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵, NR⁵R⁶ and —R^{a}—R^{b};

or R¹ is a 6-10 membered aromatic or partially saturated hydrocarbon cyclic group, optionally and independently substituted with 1-6 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵ and NR⁵R⁶;

R^{a} is a single bond or —CH₂—;

R^{b} is a 4-8 membered non-aromatic heterocyclic group, C₆₋₁₀aryl or a 5-7 membered heteroaryl group, optionally and independently substituted with 1-4 substituent(s) selected from the group consisting of halogen and C₁₋₆alkyl;

R⁵ and R⁶ are independently selected from C₁₋₆alkyl, C₁₋₆alkoxy, C₁₋₆hydroxyalkyl or a 6-membered non-aromatic heterocyclic group;

and two or more positions on R¹ are optionally bridged by a group —X— wherein X is O, CH₂, CH₂—CH₂, NR⁷, CH₂—CH₂—CH₂, CH₂—CH(CH₂—)—CH₂ or N(R⁷)—CH(CH₂—) CH₂ to form a bicyclic or tricyclic ring system, wherein R⁷ is independently selected from hydrogen or C₁₋₆alkyl and wherein said bridge may be optionally and independently substituted with one or more of C₁₋₆alkyl, cyano, CO₂NH₂, C₁₋₆hydroxyalkyl, oxo, hydroxy, C₁₋₆ alkylamino or a 6-membered non-aromatic heterocyclic group;

R² is hydrogen, C₁₋₆alkyl optionally substituted with a 4-7 membered non-aromatic heterocyclic group, or C₁₋₆haloalkyl;

R³ is hydrogen or C₁₋₆alkyl; and

R⁴ is hydrogen or C₁₋₆alkyl.

In one embodiment of the present invention, R¹ is a 5-7 membered non-aromatic hydrocarbon cyclic group optionally and independently substituted with 1-4 substituent(s) selected from the group consisting of halogen, oxo, ethylenedioxy, C₁₋₆alkyl, C₁₋₆alkoxy, C₁₋₆hydroxyalkyl, —C(O)OH, (C₁₋₆alkyl)amino, di(C₁₋₆alkyl)amino and —R^{a}—R^{b};

R^{a} is a single bond or —CH₂—; and

R^{b} is a 4-7 membered non-aromatic heterocyclic group, C₆₋₁₀aryl or a 5-7 membered heteroaryl group, optionally and independently substituted with 1-4 substituent(s) selected from the group consisting of halogen and C₁₋₆alkyl.

The present invention also provides the compound of formula (Io) or a pharmaceutically acceptable salt thereof:

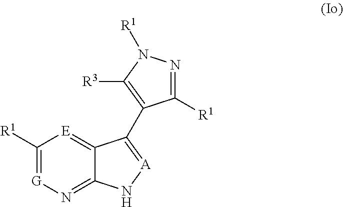

wherein

A is CH or N;

E is CH or N;

G is CH or N;

A is N when E and G are CH;

E is N when A and G are CH;

G is N when A and E are CH;

R¹ is a 5-7 membered non-aromatic hydrocarbon cyclic group optionally and independently substituted with 1-4 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵, NR⁵R⁶ and —R^{a}—R^{b};

R^{a} is a single bond or —CH₂—;

R^{b} is a 4-8 membered non-aromatic heterocyclic group, C₆₋₁₀aryl or a 5-7 membered heteroaryl group, optionally and independently substituted with 1-4 substituent(s) selected from the group consisting of halogen and C₁₋₆alkyl;

R⁵ and R⁶ are independently selected from C₁₋₆alkyl, C₁₋₆alkoxy, C₁₋₆hydroxyalkyl or a 6-membered non-aromatic heterocyclic group;

and two or more positions on R¹ are optionally bridged by a group —X— wherein X is O, CH₂, CH₂—CH₂, NR⁷, CH₂—CH₂—CH₂, CH₂—CH(CH₂—)—CH₂ or N(R⁷)—CH(CH₂—) CH₂ to form a bicyclic or tricyclic ring system, wherein R⁷ is independently selected from hydrogen or C₁₋₆alkyl and wherein said bridge may be optionally and independently substituted with one or more of C₁₋₆alkyl, cyano, CO₂NH₂, C₁₋₆hydroxyalkyl, oxo, hydroxy, C₁₋₆ alkylamino or a 6-membered non-aromatic heterocyclic group;

R² is hydrogen, C₁₋₆alkyl optionally substituted with a 4-7 membered non-aromatic heterocyclic group, or C₁₋₆haloalkyl;

R³ is hydrogen or C₁₋₆alkyl; and

R⁴ is hydrogen or C₁₋₆alkyl.

In one embodiment of the present invention, R¹ is a 5-7 membered non-aromatic hydrocarbon cyclic group optionally and independently substituted with 1-4 substituent(s) selected from the group consisting of halogen, oxo, ethylenedioxy, C₁₋₆alkyl, C₁₋₆alkoxy, C₁₋₆hydroxyalkyl, —C(O)OH, (C₁₋₆alkyl)amino, di(C₁₋₆alkyl)amino and —R^{a}—R^{b};

R^{a} is a single bond or —CH₂—; and

R^{b} is a 4-7 membered non-aromatic heterocyclic group, C₆₋₁₀aryl or a 5-7 membered heteroaryl group, optionally and independently substituted with 1-4 substituent(s) selected from the group consisting of halogen and C₁₋₆alkyl.

Preferably, R¹ is a 5-7 membered non-aromatic hydrocarbon cyclic group optionally and independently substituted with 1-4 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵, NR⁵R⁶ and —R^{a}—R^{b}, wherein R⁵, R⁶, R^{a} and R^{b} are as hereinabove defined;

or R¹ is phenyl, optionally substituted with 1-3 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵ and NR⁵R⁶, wherein R⁵ and R⁶ are as hereinabove defined.

More preferably, R¹ is a 5-7 membered non-aromatic hydrocarbon cyclic group optionally and independently substituted with 1-3 substituent(s) selected from the group consisting of halogen, oxo, ethylenedioxy, C₁₋₆alkyl, C₁₋₆alkoxy, C₁₋₆hydroxyalkyl, —C(O)OH and —R^{a}—R^{b};

R^{a} is a single bond or —CH₂—; and

R^{b} is a 4-7 membered non-aromatic heterocyclic group, C₆₋₁₀aryl or a 5-6 membered heteroaryl group, optionally and independently substituted with 1-3 substituent(s) selected from the group consisting of halogen and C₁₋₆alkyl;

or R¹ is phenyl, optionally substituted with 1-2 substituent(s) selected from the group consisting of halogen, cyano, hydroxy, oxo, C₁₋₆alkyl, C₁₋₆alkoxy, C₁₋₆hydroxyalkyl, —C(O)OH, —CONH₂, NHR⁵ and NR⁵R⁶.

Most preferably, R¹ is cyclopentyl, cyclohexyl, cycloheptyl, cyclopentenyl, cyclohexenyl, cycloheptenyl, cyclohexadienyl, each of which may optionally and independently substituted with 1-3 substituent(s) selected from the group consisting of halogen, oxo, ethylenedioxy, C₁₋₆alkyl, C₁₋₆alkoxy, C₁₋₆hydroxyalkyl and —R^{a}—R^{b};

wherein R^{a} is a single bond or —CH₂—;

wherein R^{b} is a 4-7 membered non-aromatic heterocyclic group, C₆₋₁₀aryl or a 5-6 membered heteroaryl group, optionally and independently substituted with 1-2 substituent(s) selected from the group consisting of halogen and C₁₋₆alkyl.

Especially, R¹ is cyclopentyl, cyclohexyl or cycloheptyl, optionally and independently substituted with 1-2 substituent(s) selected from the group consisting of halogen, C₁₋₄alkyl, C₁₋₄alkoxy, C₁₋₄hydroxyalkyl and —R^{a}—R^{b};

wherein R^{a} is a single bond or —CH₂—;

wherein R^{b} is a 5-7 membered non-aromatic heterocyclic group, C₆₋₁₀aryl or a 5-6 membered heteroaryl group, optionally and independently substituted with 1-2 substituent(s) selected from the group consisting of halogen and C₁₋₄alkyl.

More especially, R¹ is cyclopentyl or cyclohexyl optionally and independently substituted with 1-2 substituent(s) selected from the group consisting of fluorine, methyl, ethyl, t-butyl and methoxy, piperidinyl, fluoropiperidinyl, pyrrolidinyl, methylpiperazinyl, isopropylpiperazinyl, methyldiazepanyl, morpholinyl and oxazepanyl.

Most especially, R¹ is cyclohexyl optionally and independently substituted with 1-2 substituent(s) selected from the group consisting of methylpiperazinyl, fluoropiperidinyl, morpholinyl and oxazepanyl.

Particularly, R¹ is cyclohexyl optionally substituted with 4-methylpiperazinyl, 4-fluoropiperidinyl, morpholinyl and oxazepanyl.

When R^{b} is a 4-8 membered non-aromatic heterocyclic group that contains at least one nitrogen atom, preferably R^{b} is attached to R^{a} via the nitrogen atom.

When R¹ is a mono-substituted cyclohexyl group, preferably the substituent is at the 4-position of the cyclohexyl group.

When R¹ is a 4-substituted cyclohexyl group, preferably it has a trans configuration as illustrated in formula (II) below:

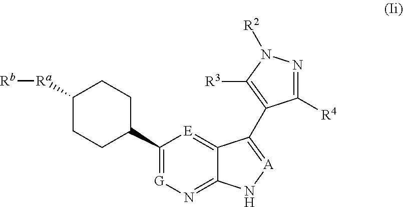

Examples of suitable R¹ groups include phenyl, cyclohexyl,

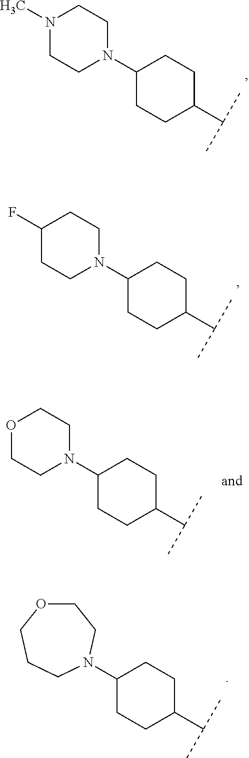

In another embodiment, R² is methyl, morpholinoethyl or trifluoromethyl. Preferably, R² is methyl.

In another embodiment, R³ is hydrogen or methyl. Preferably, R³ is hydrogen.

In another embodiment, R⁴ is hydrogen or methyl. Preferably, R⁴ is hydrogen.

One favoured group of compounds of the present invention is the compound of formula (Ia) and pharmaceutically acceptable salts thereof:

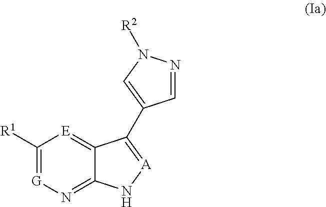

where A, E, G, R¹ and R² are as defined in relation to formula (I).

Another favoured group of compounds of the present invention is the compound of formula (Ib) and pharmaceutically acceptable salts thereof:

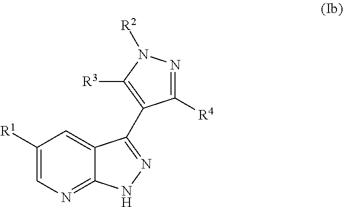

where R¹, R², R³ and R⁴ are as defined in relation to formula (I).

Another favoured group of compounds of the present invention is the compound of formula (Ic) and pharmaceutically acceptable salts thereof:

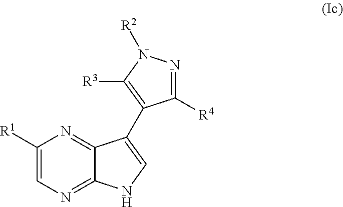

where R¹, R², R³ and R⁴ are as defined in relation to formula (I).

Another favoured group of compounds of the present invention is the compound of formula (Id) and pharmaceutically acceptable salts thereof:

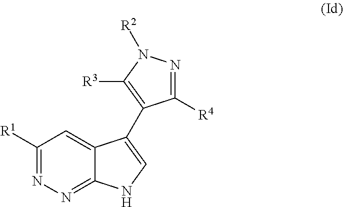

In a further aspect, the invention relates to a compound selected from the following group or a pharmaceutically acceptable salt thereof:

### EXAMPLE 1

### 5-cyclohexyl-3-(1-methyl-1H-pyrazol-4-yl)-1H-pyrazolo[3,4-b]pyridine (I-A-2)

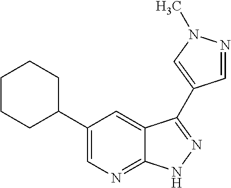

### EXAMPLE 2

### 4-((1r,4r)-4-(3-(1-methyl-1H-pyrazol-4-yl)-1H-pyrazolo[3,4-b]pyridin-5-yl)cyclohexyl)-1,4-oxazepane (I-A-4)

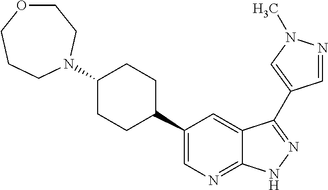

### EXAMPLE 3

### 4-((1s,4s)-4-(3-(1-methyl-1H-pyrazol-4-yl)-1H-pyrazolo[3,4-b]pyridin-5-yl)cyclohexyl)-1,4-oxazepane (I-A-5)

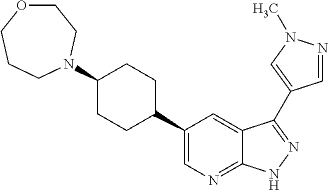

### EXAMPLE 4

### 2-cyclohexyl-7-(1-methyl-1H-pyrazol-4-yl)-5H-pyrrolo[3,2-b]pyrazine (I-E-2)

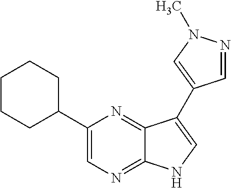

### EXAMPLE 5

### 4-((1r,4r)-4-(7-(1-methyl-1H-pyrazol-4-yl)-5H-pyrrolo[3,2-b]pyrazin-2-yl)cyclohexyl)morpholine (I-E-7)

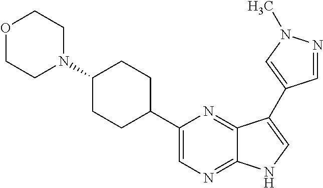

### EXAMPLE 6

### 5-(1-methyl-1H-pyrazol-4-yl)-3-phenyl-7H-pyrrolo[2,3-c]pyridazine

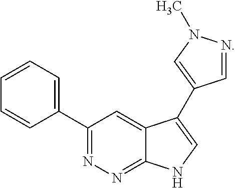

In a further aspect, the invention provides a pharmaceutical composition comprising a compound as defined herein, or a pharmaceutically acceptable salt thereof, in association with a pharmaceutically acceptable carrier.

In a further aspect, the invention provides a compound or a pharmaceutical composition as defined herein for use in medicine.

In a further aspect, the invention provides a compound or a pharmaceutical composition as defined herein for use in preventing and/or treating a neurodegenerative disorder, an inflammatory disease, an autoimmune disease and/or organ failure. Preferably, the neurodegenerative disorder is multiple sclerosis. Preferably, the inflammatory disease is multiple sclerosis. Preferably, the autoimmune disease is rheumatoid arthritis. Preferably, the organ failure is heart failure, liver failure or diabetic nephropathy.

In a further aspect, the invention provides a method for preventing and/or treating a neurodegenerative disorder (for example, multiple sclerosis), an inflammatory disease (for example, multiple sclerosis), an autoimmune disease (for example, rheumatoid arthritis) and/or organ failure (for example, heart failure, liver failure or diabetic nephropathy), which comprises administering to a mammalian animal an effective amount of a compound or a composition as defined herein.

In a further aspect, the invention provides the use of a compound or pharmaceutically salt thereof as defined herein, for the manufacture of a medicament for the prevention and/or treatment of a neurodegenerative disorder (for example, multiple sclerosis), an inflammatory disease (for example, multiple sclerosis), an autoimmune disease (for example, rheumatoid arthritis) and/or organ failure (for example, heart failure, liver failure or diabetic nephropathy).

The invention further provides a process for the manufacture of a compound of formula (I) and intermediates involved in the manufacture of a compound of formula (I). Processes for the manufacture of said compound and intermediates are described hereinafter in Reaction Schemes 1 and 2 and are illustrated by the accompanying examples.

### DETAILED DESCRIPTION OF THE INVENTION

The present invention provides the compound of formula (I) or a pharmaceutically acceptable salt thereof:

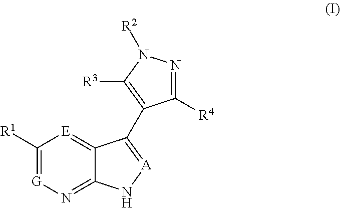

wherein

A is CH or N;

E is CH or N;

G is CH or N;

A is N when E and G are CH;

E is N when A and G are CH;

G is N when A and E are CH;

R¹ is a 5-7 membered non-aromatic hydrocarbon cyclic group optionally and independently substituted with 1-4 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵, NR⁵R⁶ and —R^{a}—R^{b};

or R¹ is a 6-10 membered aromatic or partially saturated hydrocarbon cyclic group, optionally and independently substituted with 1-6 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵ and NR⁵R⁶;

R^{a} is a single bond or —CH₂—;

R^{b} is a 4-8 membered non-aromatic heterocyclic group, C₆₋₁₀aryl or a 5-7 membered heteroaryl group, optionally and independently substituted with 1-4 substituent(s) selected from the group consisting of halogen and C₁₋₆alkyl;

R⁵ and R⁶ are independently selected from C₁₋₆alkyl, C₁₋₆alkoxy, C₁₋₆hydroxyalkyl or a 6-membered non-aromatic heterocyclic group;

and two or more positions on R¹ are optionally bridged by a group —X— wherein X is O, CH₂, CH₂—CH₂, NR⁷, CH₂—CH₂—CH₂, CH₂—CH(CH₂—)—CH₂ or N(R⁷)—CH(CH₂—) CH₂ to form a bicyclic or tricyclic ring system, wherein R⁷ is independently selected from hydrogen or C₁₋₆alkyl and wherein said bridge may be optionally and independently substituted with one or more of C₁₋₆alkyl, cyano, CO₂NH₂, C₁₋₆hydroxyalkyl, oxo, hydroxy, C₁₋₆ alkylamino or a 6-membered non-aromatic heterocyclic group;

R² is hydrogen, C₁₋₆alkyl optionally substituted with a 4-7 membered non-aromatic heterocyclic group, or C₁₋₆haloalkyl;

R³ is hydrogen or C₁₋₆alkyl; and

R⁴ is hydrogen or C₁₋₆alkyl.

In one embodiment of the present invention, R¹ is a 5-7 membered non-aromatic hydrocarbon cyclic group optionally and independently substituted with 1-4 substituent(s) selected from the group consisting of halogen, oxo, ethylenedioxy, C₁₋₆alkyl, C₁₋₆alkoxy, C₁₋₆hydroxyalkyl, —C(O)OH, (C₁₋₆alkyl)amino, di(C₁₋₆alkyl)amino and —R^{a}—R^{b};

R^{a} is a single bond or —CH₂—; and

R^{b} is a 4-7 membered non-aromatic heterocyclic group, C₆₋₁₀aryl or a 5-7 membered heteroaryl group, optionally and independently substituted with 1-4 substituent(s) selected from the group consisting of halogen and C₁₋₆alkyl.

Preferably, R¹ is a 5-7 membered non-aromatic hydrocarbon cyclic group optionally and independently substituted with 1-4 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵, NR⁵R⁶ and —R^{a}—R^{b}, wherein R⁵, R⁶, R^{a} and R^{b} are as hereinabove defined;

or R¹ is phenyl, optionally substituted with 1-3 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵ and NR⁵R⁶, wherein R⁵ and R⁶ are as hereinabove defined.

More preferably, R¹ is a 5-7 membered non-aromatic hydrocarbon cyclic group optionally and independently substituted with 1-3 substituent(s) selected from the group consisting of halogen, oxo, ethylenedioxy, C₁₋₆alkyl, C₁₋₆alkoxy, C₁₋₆hydroxyalkyl, —C(O)OH and —R^{a}—R^{b};

R^{a} is a single bond or —CH₂—; and

R^{b} is a 4-7 membered non-aromatic heterocyclic group, C₆₋₁₀aryl or a 5-6 membered heteroaryl group, optionally and independently substituted with 1-3 substituent(s) selected from the group consisting of halogen and C₁₋₆alkyl;

or R¹ is phenyl, optionally substituted with 1-2 substituent(s) selected from the group consisting of halogen, cyano, hydroxy, oxo, C₁₋₆alkyl, C₁₋₆alkoxy, C₁₋₆hydroxyalkyl, —C(O)OH, —CONH₂, NHR⁵ and NR⁵R⁶.

Most preferably, R¹ is cyclopentyl, cyclohexyl, cycloheptyl, cyclopentenyl, cyclohexenyl, cycloheptenyl, cyclohexadienyl, each of which may optionally and independently substituted with 1-3 substituent(s) selected from the group consisting of halogen, oxo, ethylenedioxy, C₁₋₆alkyl, C₁₋₆alkoxy, C₁₋₆hydroxyalkyl and —R^{a}—R^{b};

wherein R^{a} is a single bond or —CH₂—;

wherein R^{b} is a 4-7 membered non-aromatic heterocyclic group, C₆₋₁₀aryl or a 5-6 membered heteroaryl group, optionally and independently substituted with 1-2 substituent(s) selected from the group consisting of halogen and C₁₋₆alkyl.

Especially, R¹ is cyclopentyl, cyclohexyl or cycloheptyl, optionally and independently substituted with 1-2 substituent(s) selected from the group consisting of halogen, C₁₋₄alkyl, C₁₋₄alkoxy, C₁₋₄hydroxyalkyl and —R^{a}—R^{b};

wherein R^{a} is a single bond or —CH₂—;

wherein R^{b} is a 5-7 membered non-aromatic heterocyclic group, C₆₋₁₀aryl or a 5-6 membered heteroaryl group, optionally and independently substituted with 1-2 substituent(s) selected from the group consisting of halogen and C₁₋₄alkyl.

More especially, R¹ is cyclopentyl or cyclohexyl optionally and independently substituted with 1-2 substituent(s) selected from the group consisting of fluorine, methyl, ethyl, t-butyl and methoxy, piperidinyl, fluoropiperidinyl, pyrrolidinyl, methylpiperazinyl, isopropylpiperazinyl, methyldiazepanyl, morpholinyl and oxazepanyl.

Most especially, R¹ is cyclohexyl optionally and independently substituted with 1-2 substituent(s) selected from the group consisting of methylpiperazinyl, fluoropiperidinyl, morpholinyl and oxazepanyl.

Particularly, R¹ is cyclohexyl optionally substituted with 4-methylpiperazinyl, 4-fluoropiperidinyl, morpholinyl and oxazepanyl.

When R^{b} is a 4-8 membered non-aromatic heterocyclic group that contains at least one nitrogen atom, preferably R^{b} is attached to R^{a} via the nitrogen atom.

When R¹ is a mono-substituted cyclohexyl group, preferably the substituent is at the 4-position of the cyclohexyl group.

When R¹ is a 4-substituted cyclohexyl group, preferably it has a trans configuration as illustrated in formula (Ie) below:

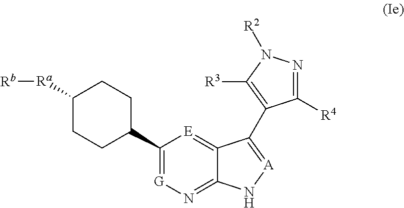

Examples of suitable R¹ groups include phenyl, cyclohexyl,

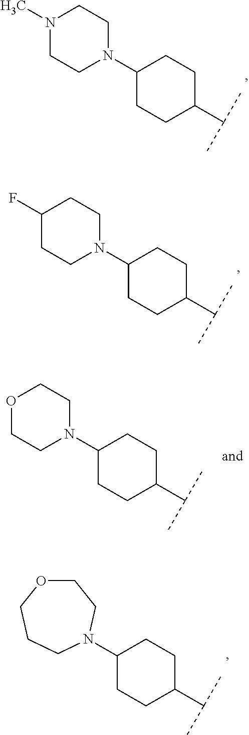

In another embodiment, R² is methyl, morpholinoethyl or trifluoromethyl. Preferably, R² is methyl.

In another embodiment, R³ is hydrogen or methyl. Preferably, R³ is hydrogen.

In another embodiment, R⁴ is hydrogen or methyl. Preferably, R⁴ is hydrogen.

The compounds of the present invention are provided for the prevention and or treatment of neurodegenerative disorders, inflammatory diseases and/or autoimmune diseases and/or organ failure.

Examples of neurodegenerative disorders are multiple sclerosis, dementia, Alzheimer's disease, Parkinson's disease, Amyotrophic Lateral Sclerosis, Huntington's disease, senile chorea, Sydenham's chorea, hypoglycaemia, head and spinal cord trauma including traumatic head injury, acute and chronic pain, epilepsy and seizures, olivopontocerebellar dementia, neuronal cell death, hypoxia-related neurodegeneration, acute hypoxia, glutamate toxicity including glutamate neurotoxicity, cerebral ischemia, dementia linked to meningitis and/or neurosis, cerebrovascular dementia, or dementia in an HIV-infected patient. Preferably, the neurodegenerative disorder is multiple sclerosis.

Examples of autoimmune diseases are multiple sclerosis, rheumatoid arthritis, systemic lupus erythematosus, glomerulonephritis, scleroderma, chronic thyroiditis, Graves' disease, autoimmune gastritis, diabetes, autoimmune haemolytis anaemia, autoimmune neutropenia, thrombocytopenia, atopic dermatitis, chronic active hepatitis, myasthenia gravis, multiple sclerosis, ulcerative colitis, Crohn's disease, psoriasis or graft vs host disease. Preferably, the autoimmune disease is rheumatoid arthritis.

Examples of inflammatory diseases are asthma, autoimmune diseases (including multiple sclerosis, systemic Lupus erythematosus), chronic inflammation, chronic prostatitis, glomerulonephritis, hypersensitivity, inflammatory bowel disease, pelvic inflammatory disease, reperfusion injury, rheumatoid arthritis, transplant rejection and vasculitis.

It will be appreciated that an inflammatory disease is a disease accompanied by a cascade of biochemical events including the local vascular system, the immune system and various cells within the injured tissues, e.g. brain, spinal cord, synovial joints, organ systems (heart, liver, kidney lung, gut) and soft tissue, (muscle, skin) etc. For the purposes of the present invention, inflammation can either be acute or chronic. The inflammatory diseases for the present invention include those which involve the immune system (i.e. as demonstrated in allergic reaction and some myopathies). The inflammatory diseases for the present invention further include non-immune diseases with aetiological origins in inflammatory processes including cancer, atherosclerosis and ischaemic heart disease.

The compounds of the present invention are further provided for the prevention and/or treatment of organ failure, particularly of the heart, liver or kidneys. Examples of organ failure are chronic or acute cardiac failure, cardiac hypertrophy, dilated, hypertrophic or restrictive cardiomyopathy, acute myocardial infarction, post-myocardial infarction, acute or chronic myocarditis, diastolic dysfunction of the left ventricle, systolic dysfunction of the left ventricle, hypertension and nephropathy and nephritis as complications thereof, diabetic nephropathy, endothelial dysfunction, arteriosclerosis or post-angioplasty restenosis. The invention particularly relates to the prevention and/or treatment of diabetic nephropathy.

The compounds of the present invention are further provided for the prevention and/or treatment of chronic rheumatoid arthritis, osteoarthritis, gout, chronic obstructive pulmonary disease, asthma, bronchitis, cystic fibrosis, inflammatory bowel disease, irritable colon syndrome, mucous colitis, ulcerative colitis, Crohn's disease, gastritis, oesophagitis, eczema, dermatitis, hepatitis, glomerulonephritis, ophthalmic diseases, diabetic retinopathy, diabetic macular oedema, diabetic nephropathy, diabetic neuropathy, obesity, psoriasis, cancer, cerebral apoplexy, cerebrovascular disorder, an ischemic disorder of an organ selected from the heart, kidney, liver and brain, ischemia reperfusion injury, endotoxin shock or rejection in transplantation.

The term “halogen” used herein means a fluorine atom, a chlorine atom, a bromine atom, an iodine atom and the like.

The term “C₁₋₆alkyl” used herein means an alkyl group that is a straight or branched chain with 1 to 6 carbons. The alkyl group therefore has 1, 2, 3, 4, 5 or 6 carbon atoms. Examples of “C₁₋₆alkyl” include methyl, ethyl, n-propyl, iso-propyl, n-butyl, iso-butyl, sec-butyl, tert-butyl, n-pentyl, 1,1-dimethylpropyl, 1,2-dimethylpropyl, 2,2-dimethylpropyl, 1-ethylpropyl, n-hexyl, 1-ethyl-2-methylpropyl, 1,1,2-trimethylpropyl, 1-ethylbutyl, 1-methylbutyl, 2-methylbutyl, 1,1-dimethylbutyl, 1,2-dimethylbutyl, 2,2-dimethylbutyl, 1,3-dimethylbutyl, 2,3-dimethylbutyl, 2-ethylbutyl, 2-methylpentyl, 3-methylpentyl and the like.

The term “C₁₋₆haloalkyl” used herein means a C₁₋₆alkyl group as described above substituted with 1, 2 or 3 halogen atom(s). Examples of “C₁₋₆haloalkyl” include fluoromethyl, difluoromethyl, trifluoromethyl, fluoroethyl, difluoroethyl, trifluoroethyl, chloromethyl, bromomethyl, iodomethyl and the like.

The term “C₁₋₆alkoxy” used herein means an oxy group that is bonded to the previously defined “C₁₋₆alkyl”. Examples of “C₁₋₆alkoxy” include methoxy, ethoxy, n-propoxy, iso-propoxy, n-butoxy, iso-butoxy, sec-butoxy, tert-butoxy, n-pentyloxy, iso-pentyloxy, sec-pentyloxy, n-hexyloxy, iso-hexyloxy, 1,1-dimethylpropoxy, 1, 2-1dimethylpropoxy, 2,2-dimethylpropoxy, 2-methylbutoxy, 1-ethyl-2-methylpropoxy, 1,1,2-trimethylpropoxy, 1,1-dimethylbutoxy, 1,2-dimethylbutoxy, 2,2-dimethylbutoxy, 2,3-dimethylbutoxy, 1,3-dimethylbutoxy, 2-ethylbutoxy, 2-methylpentyloxy, 3-methylpentyloxy and the like.

The term “(C₁₋₆alkyl)amino” used herein means an amino group which is substituted with a C₁₋₆alkyl group as described above.

The term “di(C₁₋₆alkyl)amino” used herein means an amino group which is substituted with two C₁₋₆alkyl group as described above.

The term “5-7 membered non-aromatic hydrocarbon cyclic group” used herein means 5-7 membered cycloalkyl group, 5-7 membered cycloalkenyl group and 5-7 membered cycloalkadienyl group. The non-aromatic hydrocarbon cyclic group therefore has 5, 6 or 7 ring members. Examples of “5-7 membered non-aromatic hydrocarbon cyclic group” include cyclopentyl, cyclohexyl, cycloheptyl, cyclopentenyl, cyclohexenyl, cycloheptenyl, cyclopentadienyl, cyclohexadienyl, cycloheptadienyl, bornane, adamantane, 7-oxabicyclo[2.2.1]hept-2,3-ene, 7-oxabicyclo[2.2.1]heptane and 7-aminobicyclo[2.2.1]hept-2,3-ene.

The non-aromatic hydrocarbon cyclic group may be provided as a bicyclic or tricyclic ring system having two or more shared or common atoms. In this case, the non-aromatic hydrocarbon cyclic group comprises a bridging moiety having one or more atoms selected from C, N, O or S, said bridging moiety connecting the two or more shared or common atoms.

Preferably, the non-aromatic hydrocarbon cyclic group is a six membered cycloalkyl group or a six membered cycloalkenyl group with a bridging moiety selected from —CH₂—, —O—, —N—, —(CH₂)₃—, —CH₂—CH₂—N—.

The bridging moiety can be attached to two shared or common atoms which are adjacent to each other on the non-aromatic hydrocarbon cyclic group or which are separated by one, two or three ring atoms.

Examples include bornane, norbornane, adamantane, 7-oxabicyclo[2.2.1]hept-2,3-ene, 7-oxabicyclo[2.2.1]heptane and 7-aminobicyclo[2.2.1]hept-2,3-ene.

The non-aromatic hydrocarbon cyclic group may be optionally and independently substituted at any available position on the ring atoms and/or bridging atoms with 1 to 4 substituent(s) selected from the group consisting of halogen, oxo, C₁₋₆alkyl, C₁₋₆hydroxyalkyl, —CONH₂, hydroxy, C₁₋₆alkylamino and a 6-membered non-aromatic heterocyclic group. Preferably, the substituent(s) are selected from the group consisting of methyl, CN, CO₂NH₂, CH₂—OH, ═O, OH, NHMe and a 6-membered non-aromatic heterocyclic group comprising two nitrogen atoms. When the non-aromatic hydrocarbon cyclic group is substituted on a bridging N-atom, the substituent is preferably hydrogen or C₁₋₆alkyl, more preferably hydrogen, methyl, ethyl or propyl.

The term “4-8 membered non-aromatic heterocyclic group” used herein means heterocyclic group, which has no aromaticity and the number of atoms forming the ring is 4, 5, 6, 7 or 8, containing one or more species of heteroatom selected from the group consisting of nitrogen, sulfur and oxygen. Examples of “4-8 membered non-aromatic heterocyclic group” include azetidinyl, pyrrolidinyl, imidazolidinyl, pyrazolidinyl, piperidinyl, piperazinyl, morpholinyl, tetrahydropyranyl, dioxanyl, diazepanyl, oxazepanyl, oxazocanyl and the like.

The term “C₆₋₁₀aryl” used herein means an aryl group constituted by 6, 7, 8, 9 or 10 carbon atoms and includes condensed ring groups such as monocyclic rings, bicyclic rings and the like. Examples of “C₆₋₁₀aryl” include phenyl, indenyl, naphthyl, azulenyl and the like. It should be noted that condensed rings such as indanyl and tetrahydronaphthalenyl are also included in the aryl group.

The term “5-7 membered heteroaryl group” used herein means a monocyclic heteroaryl group, in which the number of atoms forming the ring is 5, 6 or 7, containing one or more species of heteroatom selected from the group consisting of nitrogen, sulfur and oxygen. Examples of “5-7 membered heteroaryl group” include 1) pyrrolyl, pyridyl, pyridazinyl, pyrimidinyl, pyrazinyl, triazolyl, tetrazolyl, pyrazolyl, imidazolyl and the like as a nitrogen-containing heteroaryl group; 2) thienyl and the like as a sulfur-containing heteroaryl group; 3) furyl, pyranyl and the like as an oxygen-containing heteroaryl group; and 4) thiazolyl, isothiazolyl, isoxazolyl, furazanyl, oxazolyl, oxadiazolyl, pyrazolo-oxazolyl, imidazothiazolyl, furopyrrolyl, pyridooxazinyl and the like as a heteroaryl group containing two or more different species of heteroatoms.

JNK inhibitory compounds of formula (I) as defined above have significant in vitro activity.

Specifically, the present invention provides one or more of the following compounds:

### EXAMPLE 1

### 5-cyclohexyl-3-(1-methyl-1H-pyrazol-4-yl)-1H-pyrazolo[3,4-b]pyridine (I-A-2)

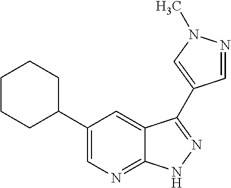

### EXAMPLE 2

### 4-((1 r,4r)-4-(3-(1-methyl-1H-pyrazol-4-yl)-1H-pyrazolo[3,4-b]pyridin-5-yl)cyclohexyl)-1,4-oxazepane (I-A-4)

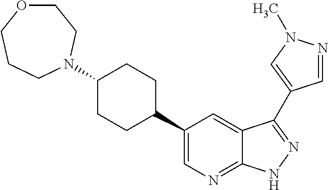

### EXAMPLE 3

### 4-((1s,4s)-4-(3-(1-methyl-1H-pyrazol-4-yl)-1H-pyrazolo[3,4-b]pyridin-5-yl)cyclohexyl)-1,4-oxazepane (I-A-5)

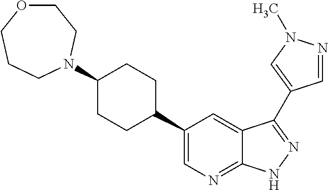

### EXAMPLE 4

### 2-cyclohexyl-7-(1-methyl-1H-pyrazol-4-yl)-5H-pyrrolo[3,2-b]pyrazine (I-E-2)

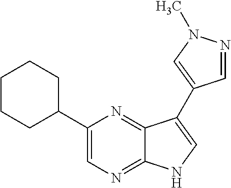

### EXAMPLE 5

### 4-((1r,4r)-4-(7-(1-methyl-1H-pyrazol-4-yl)-5H-pyrrolo[3,2-b]pyrazin-2-yl)cyclohexyl)morpholine (I-E-7)

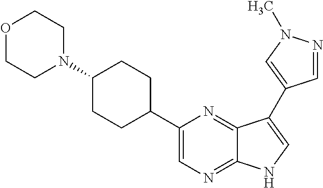

### EXAMPLE 6

### 5-(1-methyl-1H-pyrazol-4-yl)-3-phenyl-7H-pyrrolo[2,3-c]pyridazine

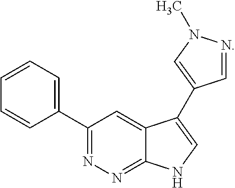

The structural formula of the compound may be described to represent a given isomer for the sake of convenience; however, all isomers of the compound that may occur structurally such as an geometric isomer, an optical isomer, a stereoisomer and a tautomer are included in the present invention, and there is no limitation to the formula described for the sake of convenience, regardless of whether it is an isolated isomer (for instance, an enantiomer), or a mixture of isomers (for instance, a racemic mixture).

When the compound according to the present invention is obtained in free form, it can be converted into a salt or a hydrate thereof by a conventional method.

Herein, there is no limitation on the “salt” according to the present invention as long as it forms a salt with the compound according to the present invention, and is pharmacologically acceptable. The preferred examples of the salt include hydrohalogenates (for instance, hydrochloride, hydrobromide, hydroiodide and the like), inorganic acid salts (for instance, sulfate, nitrate, perchlorate, phosphate, carbonate, bicarbonate and the like), organic carboxylic acid salts (for instance, acetate, maleate, tartrate, fumarate, citrate and the like), organic sulfonic acid salts (for instance, methanesulfonate, ethanesulfonate, benzenesulfonate, toluenesulfonate, camphorsulfonate and the like), amino acid salt (for instance, aspartate, glutamate and the like), quaternary ammonium salts, alkaline metal salts (for instance, sodium, potassium and the like), alkaline earth metal salts (magnesium, calcium and the like) and the like. In addition, hydrochloride, sulfate, methanesulfonate, acetate and the like are preferable as a “pharmacologically acceptable salt” of compounds according to the present invention.

Further, when the compound according to the present invention may comprise various isomers (for instance, the geometric isomer, the optical isomer, the rotational isomer, the tautomer and the like), it can also be purified into a single isomer by means of a conventional separation method, for instance, recrystallization, optical resolution such as diastereomeric salt method, enzyme fractionation method, various chromatographic methods (for instance, thin layer chromatography, column chromatography, glass chromatography and the like). However, a single isomer herein includes not only the isomer having 100% purity, but also the isomer containing non-target isomers still remaining after undergoing conventional purification operation. In addition, when using the compound according to the present invention as a raw material for a medicinal drug, the single isomer mentioned above may be used, or a mixture of isomers in any proportions may be used.

Crystal polymorphism may exist for the compound according to the present invention, salts thereof, or hydrates thereof; however, all the polymorphic crystals thereof are included in the present invention. Crystal polymorphism may exist for a single isomer or a mixture, and both are included in the present invention.

In addition, a compound still demonstrating the desired pharmacological activity after the compound according to the present invention has been subjected to metabolism such as oxidation and hydrolysis in vivo is also included in the present invention.

Furthermore, a compound which when subjected to metabolism such as oxidation, reduction and hydrolysis in vivo, generates the compound according to the present invention, a so-called prodrug, is also included in the present invention.

The present invention also includes isotopically-labelled compounds, which are identical to the compounds of formula (I), except that one or more atoms are replaced by an atom having an atomic mass or mass number different from the atomic mass or mass number usually found in nature. Examples of isotopes that can be incorporated into compounds of the invention include isotopes of hydrogen, carbon, nitrogen, oxygen, phosphorous, fluorine, iodine, and chlorine, such as ²H, ³H, ¹¹C, ¹⁴C, ¹⁸F, ³⁵S, ¹²³I and ¹²⁵I.

Compounds of the present invention and pharmaceutically acceptable derivatives (e.g. salts) of said compounds that contain the aforementioned isotopes and/or other isotopes of other atoms are within the scope of the present invention. Isotopically-labelled compounds of the present invention, for example those into which radioactive isotopes such as ³H and/or ¹⁴C are incorporated, are useful in drug and/or substrate tissue distribution assays. ³H and ¹⁴C are considered useful due to their ease of preparation and detectability. ¹¹C and ¹⁸F isotopes are considered useful in PET (positron emission tomography), and ¹²⁵I isotopes are considered useful in SPECT (single photon emission computerized tomography), all useful in brain imaging. Substitution with heavier isotopes such as ²H can afford certain therapeutic advantages resulting from greater metabolic stability, for example increased in vivo half-life or reduced dosage requirements and, hence, are considered useful in some circumstances. Isotopically labelled compounds of formula (I) of this invention can generally be prepared by carrying out the procedures disclosed in the Schemes and/or in the Examples below, by substituting a readily available isotopically labelled reagent for a non-isotopically labelled reagent.

The compound according to the present invention can be provided as a pharmaceutical composition. The pharmaceutical composition may additionally comprise a pharmaceutically acceptable excipient for example a pharmaceutically acceptable carrier and/or a pharmaceutically acceptable diluent. Suitable carrier and/or diluents are well known in the art and include pharmaceutical grade starch, mannitol, lactose, magnesium stearate, sodium saccharin, talcum, cellulose, glucose, sucrose (or other sugar), magnesium carbonate, gelatin oil, alcohol, detergents, emulsifiers or water (preferably sterile). The composition may be a mixed preparation of a composition or may be a combined preparation for simultaneous, separate or sequential use (including administration). The composition may be in any suitable form, depending on the intended method of administration. It may for example be in the form of tablet, capsule or liquid for oral administration, or of a solution or suspension for administration parenterally.

The pharmaceutical composition optionally includes one or more other agents for the treatment of neurodegenerative disorders, inflammatory disease, autoimmune disease or organ failure.

The compound according to the present invention, a salt thereof or a hydrate thereof can be formulated by a conventional method. Examples of the preferred dosage forms include a tablet, powder, subtle granule, granule, coated tablet, capsule, syrup, troche, inhalant, suppository, injectable, ointment, ophthalmic ointment, eye drop, nasal drop, ear drop, cataplasm, lotion and the like. For formulation, a diluent, binder, disintegration agent, lubricant, colorant and flavoring agent may be used in general, and as necessary, additives such as a stabilizer, emulsifier, absorption enhancer, surfactant, pH adjuster, antiseptic agent, and an antioxidant can be used. In addition, formulation is also possible by combining ingredients that are used in general as raw materials of pharmaceutical formulation, by the conventional method. Examples of these ingredients include (1) soybean oil, animal oil such as beef tallow and synthetic glyceride; (2) hydrocarbon such as liquid paraffin, squalane and solid paraffin; (3) an ester oil such as octyldodecylmyristate and isopropylmyristate; (4) higher alcohol such as cetostearylalcohol and behenyl alcohol; (5) a silicon resin; (6) a silicon oil; (7) a surfactant such as polyoxyethylene fatty acid ester, sorbitan fatty acid ester, glycerin fatty acid ester, polyoxyethylene sorbitan fatty acid ester, polyoxyethylene hardened castor oil and polyoxyethylene polyoxypropylene block co-polymer; (8) a water-soluble polymer such as hydroxyethyl cellulose, polyacrylic acid, carboxyvinyl polymer, polyethyleneglycol, polyvinylpyrrolidone and methyl cellulose; (9) lower alcohol such as ethanol and isopropanol; (10) multivalent alcohol such as glycerin, propylene glucol, dipropylene glycol and sorbitol; (11) a sugar such as glucose and cane sugar; (12) an inorganic powder such as anhydrous silicic acid, magnesium aluminium silicate and aluminium silicate; and (13) purified water and the like.

Among the aforementioned additives, use can be made of 1) lactose, corn starch, sucrose, glucose, mannitol, sorbitol, crystalline cellulose, silicon dioxide and the like as a diluting agent; 2) polyvinyl alcohol, polyvinyl ether, methyl cellulose, ethyl cellulose, gum arabic, traganth, gelatine, shellac, hydroxypropyl cellulose, hydroxypropylmethyl cellulose, polyvinylpyrrolidone, polypropyleneglycol, polyoxyethylene block co-polymer, meglumine, calcium citrate, dextrin, pectin and the like as a binder; 3) a starch, agar, gelatine powder, crystalline cellulose, calcium carbonate, sodium bicarbonate, calcium citrate, dextrin, pectin, calcium carboxymethylcellulose and the like as a disintegration agent; 4) magnesium stearate, talc, polyethyleneglycol, silica, hardened plant oil and the like as a lubricant; 5) a colorant, as long as addition thereof to a pharmaceutical drug is authorized, as a colorant; 6) cocoa powder, menthol, fragrance, peppermint oil and cinnamon powder as a flavoring agent; and 7) an antioxidant whose addition to a pharmaceutical drug is authorized, such as ascorbic acid and α-tocophenol as an antioxidant.

The compound of the invention will normally be administered in a daily dosage regimen (for an adult patient) of, for example, an oral dose of between 1 mg and 2000 mg, preferably between 30 mg and 1000 mg, for example between 10 and 250 mg or an intravenous, subcutaneous, or intramuscular dose of between 0.1 mg and 100 mg, preferably between 0.1 mg and 50 mg, for example between 1 and 25 mg of the compound of the formula (I) or a pharmaceutically acceptable salt thereof calculated as the free base, the compound being administered 1 to 4 times per day. Suitably, the compound will be administered for a period of continuous therapy, for example for a week or more.

### General Procedure

The method for preparation of a compound of formula (I) will be described below.

The compound for formula (I) can be obtained by the methods represented by the following Reaction Schemes 1 and 2 or methods equivalent thereto. Each reference symbol in the compounds shown in the following Reaction Schemes 1 and 2 has the same meaning as defined above. The compounds shown in the reaction schemes include salts formed from the compounds and examples of the salts include the same ones as the salts of the compound of formula (I) and the like.

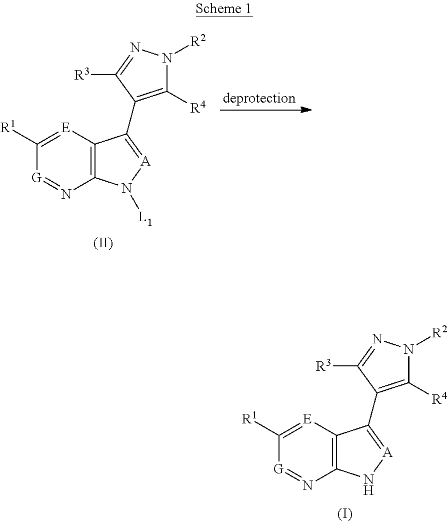

wherein A, E, G, R¹, R², R³ and R⁴ have the same meanings as described above, and L¹ is a suitable nitrogen protecting group. The conditions for the removal of the L¹ group will depend on the exact nature of the L¹ group. For example, when L¹ is phenylsulfonyl, the compound of formula (I) can be produced by the treatment of the compound of formula (II) under basic conditions, for instance using sodium hydroxide in water/ethanol.

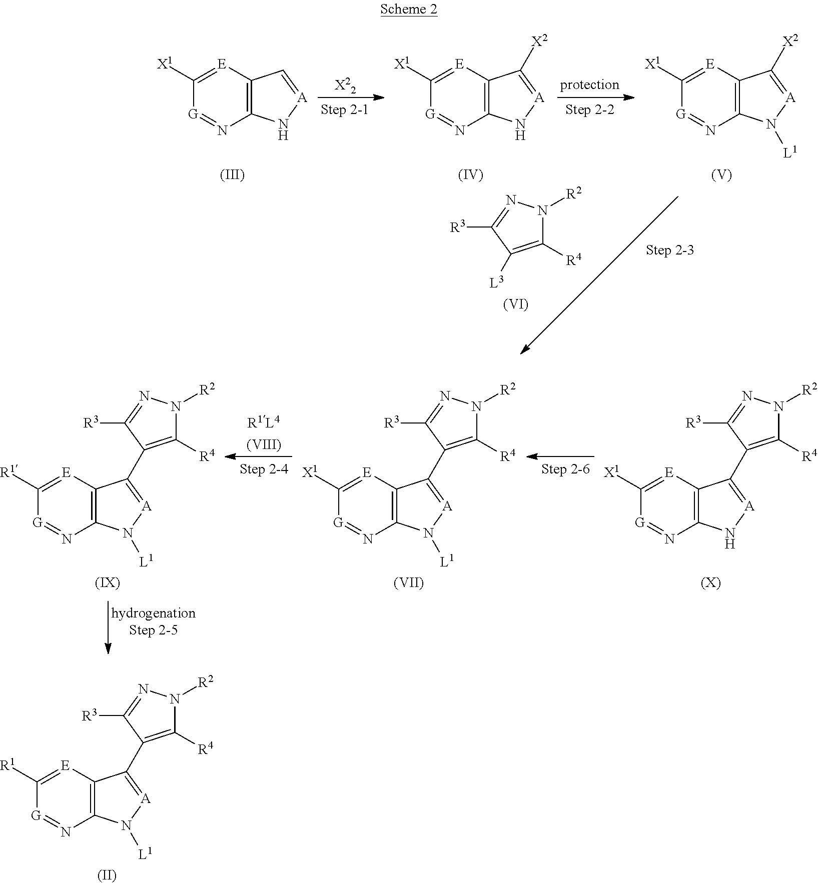

wherein A, E, G, R¹, R², R³, R⁴ and L¹ have the same meanings as described above, R^{1′} is a suitable precursor to R¹ that can be transformed into R¹ by hydrogenation, X¹ is halogen, suitably bromine, and X² is halogen, suitably iodine or bromine. L³ and L⁴ are suitable residues that can take part in a palladium-catalyzed coupling, such as a boronic acid or ester (for the Suzuki coupling), a trialkylstannyl derivative (for Stifle coupling) or a silyl group (for the Hiyama reaction). The Suzuki reaction is preferred, using a residue such as pinacolborane, i.e. B(OCMe₂)₂.

### Step 2-1

Iodination of pyrrolopyrazines (E=N) using IC1 has been disclosed in WO2006/058074. Iodination of azaindazoles (A=N) has been described in Bioorg. Med. Chem. Lett. 2007, 17, 1243. However, this reaction can also be carried out in a way analogous to that used for halogenation of 7-azaindoles, as disclosed in WO2004/78756. In particular, X²=I can be introduced by direct action of I₂ on the compound of formula (III) in the presence of a strong base such as sodium hydroxide or potassium hydroxide in anhydrous solvent such as DMF or 1,4-dioxane.

### Step 2-2

Protection of pyrrolopyrazines (E=N) with the phenylsulfonyl group (L¹=PhSO₂) has been disclosed in WO2006058074. Synthesis of the compound of formula (V) (X¹=Br, X²=I, L¹=BOC) has been described in Bioorg. Med. Chem. Lett. 2007, 17, 1243. Protection of the compound of formula (IV) can also be conducted by applying the methods used for 7-azaindoles, which have been disclosed in WO200478756 and EP1749829.

### Step 2-3

The compound of formula (VII) can be produced by coupling the compound of formula (V) with the compound of formula (VI) in the presence of a metal catalyst as disclosed in WO2004/078756 and WO2006/015123. Suitable coupling reactions include those by Stille, Suzuki, Hiyama and the like. The Stille reaction can be carried out according to Stille (Angew. Chem., Int.ed, Engl. 1986, 25, 508); Mitchell (Synthesis, 1992, 803) or Littke et al. (J. Am. Chem. Soc. 2002, 124, 6343). The Suzuki coupling can be carried out according to Suzuki (Pure Appl. Chem. 1991, 63, 419) or Littke et al. (J. Am. Chem. Soc. 2000, 122, 4020). The Hiyama reaction can be carried out according to Hatanaka et al. (J. Org. Chem. 1988, 53, 918), Hatanaka et al. (Synlett, 1991, 845), Tamao et al. (Tetrahedron Lett. 1989, 30, 6051), or Denmark et al. (Org. Lett. 2000, 2, 565, ibid. 2491).

### Step 2-4

The compound of formula (IX) can be produced by coupling the compound of formula (VII) with the compound of formula (VIII) in the presence of a metal catalyst using the methods disclosed for the 7-azaindole system in WO2004/078756 and WO2004/101565. Suitable coupling reactions include known coupling reactions such as the Stille reaction, the Suzuki coupling, the Hiyama reaction and the like as discussed above for Step 2-3.

### Step 2-5

Hydrogenation of the compound of formula (IX) can be carried out under standard conditions for reduction of a double bond using gaseous hydrogen and palladium catalyst such as Pd(OH)₂. For example, compound (IX) which contains an unsaturated ring can be reduced to form compound (II) which contains a saturated ring. The reduction can be accomplished by using hydrogen gas over a suitable catalyst such as palladium, palladium hydroxide, platinum, or rhodium.

In particular, the reduction of cyclohexenyl derivative (IX) may produce a mixture of (II-trans) and (II-cis) as shown below

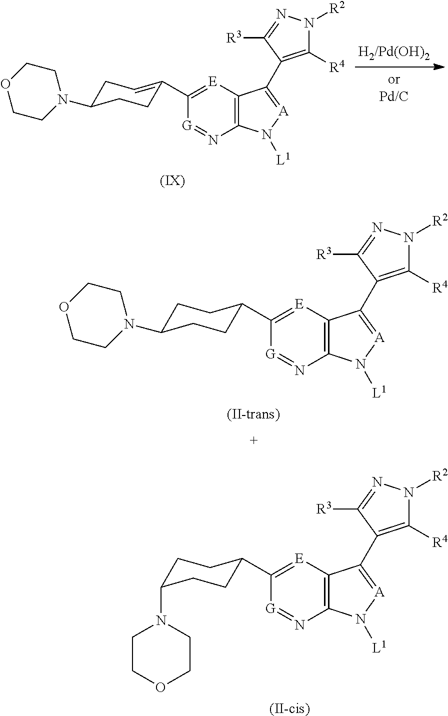

Such mixture, if needed, can be separated using chromatographic methods well known in the art. Alternatively, the cis isomers such as (II-cis) can be converted into the more thermodynamically stable trans-isomers such as (II-trans) using a free-radical method developed by Bertrand et al. (J. Org. Chem. 2006, 71, 7288).

### Step 2-6

This step may be needed if reaction carried out in Step 2-3 is accompanied by spontaneous loss of protecting group L¹. In such case, the compound of formula (X) may be protected again using the methods described in Step 2-2.

### EXAMPLES

The present invention will be described in more detail with reference to examples which however shall not be construed as limiting the scope of the invention thereto.

The examples set out below refer to the preparation of compounds falling within the scope of formula (I) which are specific examples of compounds falling within the scope of the invention.

All solvents were obtained from commercial sources (Sigma-Aldrich) and were used without further purification. With the exception of routine deprotection and coupling steps, reactions were carried out under an atmosphere of nitrogen. Organic extracts were dried over magnesium sulfate and were concentrated (after filtration of the drying agent) on rotary evaporators operating under reduce pressure. Flash chromatography was carried out on silica gel following published procedure (W. C. Still et al., J. Org. Chem. 1978, 43, 2923) or on commercial flash chromatography systems (Biotage corporation and Jones Flashmaster II) utilising pre-packed columns.

Reagents were usually obtained directly from commercial suppliers (and used as supplied) but a limited number of compounds from in-house corporate collections were utilised. In the latter case, the reagents are readily accessible using routine synthetic steps that are either reported in the scientific literature or are known to those skilled in the art.

¹H NMR spectra were recorded on Bruker Avance 400 series spectrometer operating at (reported) frequency of 400 MHz. Chemical shifts (8) for signals corresponding to non-exchangeable protons (and exchangeable protons when visible) are recorded in parts per million (ppm) relative to tetramethylsilane and are measured using the residual solvent peak as reference. Signals are listed in the order: multiplicity (s, singlet; d, doublet; t, triplet; q, quartet; m, multiplet; b, broad, and combinations thereof); coupling constant(s) in hertz (Hz); number of protons. Mass spectral (MS) data were obtained on a mass detector of Agilent 1100 LCMS system operating in positive (ES⁺) ionisation mode and results are reported as the ratio of mass over charge (m/z) for the parent ion only. Preparative scale LCMS separations were carried out on the Agilent 1100 or on a Gilson preparative system. In all cases, compounds were eluted with linear gradients of water and MeCN both containing 0.1% acetic acid using flow rate of about 80 mL/min.

The following abbreviations are used in the examples, the schemes and the tables: Ac, (acetyl, CH₃CO), BOC (tert-butoxycarbonyl), Bu (butyl), DMAP (4-N,N-dimethylaminopyridine), DMF (dimethylformamide), Et₃N (triethylamine), EtOAc (ethyl acetate), EtOH (ethanol), h (hour), LCMS (mass-detected liquid chromatography), LiHMDS (lithium hexamethyldisilazide), Me (methyl), MeCN (acetonitrile), MeOH (methanol), MHz (megahertz), min (minute), MS (mass spectrum), NaHMDS (sodium hexamethyldisilazide), NMR (nuclear magnetic resonance), Ph (phenyl), PhMe (toluene), PTLC (preparative thin layer chromatography), quant (quantitative), RT (room temperature), SGC (silica gel chromatography), THF (tetrahydrofuran), TLC (thin layer chromatography), v (volume), v/v (volume/volume; volume ratio).

### Synthetic Methods for Synthesis of Compounds of the Invention

### General Procedure for the Deprotection of Diazaindoles

### Procedure A: Removal of the Phenylsulfonyl Group

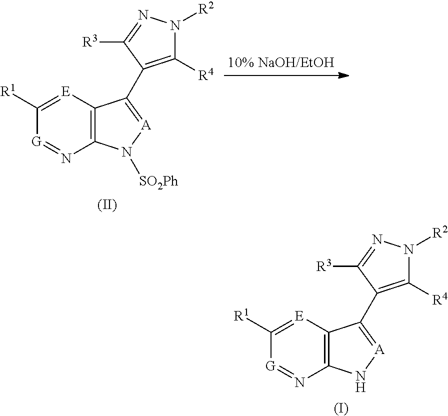

wherein A, E, G, R¹, R², R³ and R⁴ have the same meanings as described above. The diazaindole (II) (1 mmol) was dissolved in EtOH (10 mL). 10% NaOH (5 mL) was added and the reaction was heated to 80° C. for 40 mins. It was allowed to cool and a saturated solution of NaHCO₃ (10 mL) was added. It was then extracted with EtOAc (3×20 mL) and the combined organic extracts were dried over MgSO₄ and concentrated. The crude product was purified by SGC using a suitable solvent as eluent or by PTLC using a suitable solvent as the eluent or by LCMS (column LUNA 10μ C18(2) 00G-4253-V0 250×50 mm) using water-MeCN (0.1% AcOH) as eluent (in gradient; flow 80 mL/min).

### Procedure B: Removal of the Silyl Group

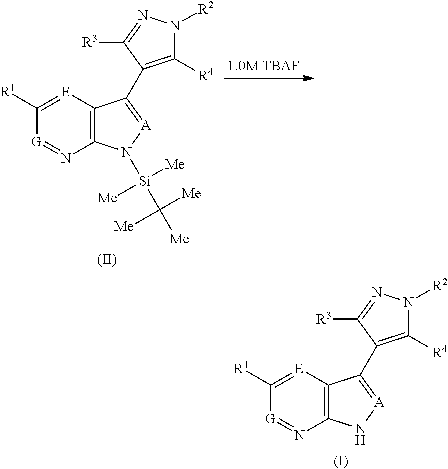

wherein A, E, G, R¹, R², R³ and R⁴ have the same meanings as described above. To a stirred solution of the silyl-protected diazaindole (II) (11.4 mmol) in THF (50 mL) was added 1 M tetrabutylammonium fluoride in THF (22.7 mL, 22.7 mmol). After 75 min the mixture was concentrated and the residue was purified by SGC using a suitable solvent as eluent or by PTLC using a suitable solvent as the eluent or by LCMS (column LUNA 10μ C18(2) 00G-4253-V0 250×50 mm) using water-MeCN (0.1% AcOH) as eluent (in gradient; flow 80 mL/min) to afford the diazaindole (I). Yield about 80%.

### Procedure C: Removal of the Silyl Group

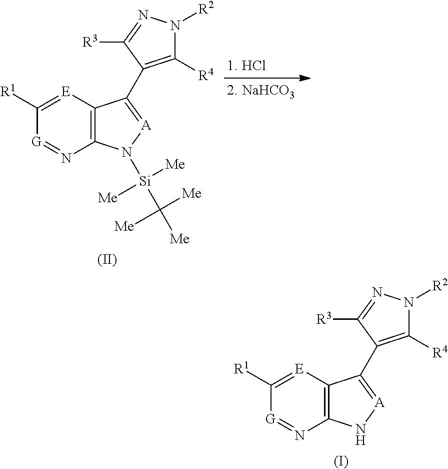

wherein A, E, G, R¹, R², R³ and R⁴ have the same meanings as described above. Concentrated aqueous HCl (1 mL) was added to a solution of silyl-protected diazaindole (II) (0.28-0.9 mmol) in MeOH (10 mL) and the reaction mixture was stirred at RT for 15-30 min. The mixture was then added to saturated aqueous NaHCO₃ (50 mL) and extracted with EtOAc (2×40 mL). The combined organic portions were dried (MgSO₄), concentrated, and purified by trituration with Et₂O (5 mL) to afford diazaindole (I) as a white powder (50-95%).

### General Procedure for the Hydrogenation of Diazaindoles Containing a Partially Unsaturated Ring at C(5)

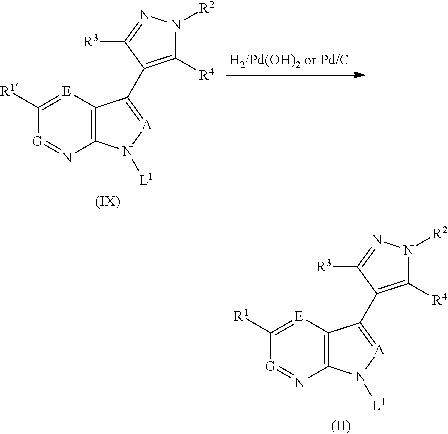

wherein A, E, G, R¹, R², R³ and R⁴ have the same meanings as described above, R¹ is a suitable precursor to R¹ that can be transformed into R¹ by hydrogenation. The compound of formula (IX) (1 mmol) was dissolved in a suitable solvent (MeOH or a mixture of MeOH and CH₂Cl₂ or EtOAc to improve solubility) (10-30 mL). Pd(OH)₂ (0.1-0.3 mmol) (20% on C, wet, Degussa type) or Pd/C (0.25-0.50 mmol) (10% on C, wet Degussa type E101) was added in one portion. The reaction was stirred under hydrogen for 1-7 days. The reaction mixture was filtered through a small pad of Celite and washed with copious amount of MeOH. The solvent was removed to give the product (II) which was taken forward crude.

### General Procedures for the Reductive Amination Involving Amines and Diazaindoles Containing Keto Functionality

### Procedure A

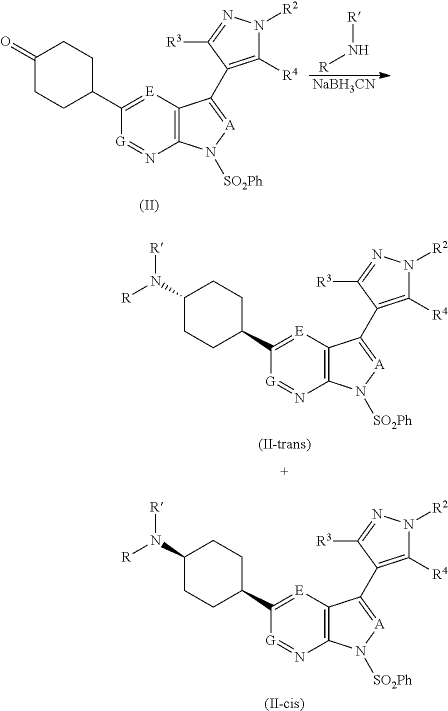

wherein A, E, G, R², R³ and R⁴ have the same meanings as described above, R and R′ are independently hydrogen or C₁₋₆alkyl, or R and R′, together with the nitrogen atom they are bonded to, form a 4-8 membered ring optionally substituted with halogen or C₁₋₆alkyl. Ketone (II) (1 mmol) was added at RT over 5 min to a solution of secondary amine R′RNH hydrochloride (6 mmol) in dry MeOH (10 mL) under nitrogen and the mixture was then stirred for 5 min at RT. When using free amine, the corresponding hydrochloride salt was prepared in situ by adding dropwise 1.25 M solution of HCl in MeOH (2 mmol) and stirring at RT for 5 min. Solid NaCNBH₃ (2 mmol) was added in one portion. The reaction was then stirred at RT overnight. Saturated solution of NaHCO₃ (30 mL) was added and the reaction mixture was extracted with EtOAc (4×35 mL). The combined organic extracts were dried over MgSO₄ and concentrated. The crude product was purified by SGC using a suitable solvent as eluent or by PTLC using a suitable solvent as the eluent or by LCMS (column LUNA 10μ C18(2) 00G-4253-V0 250×50 mm) using water-MeCN (0.1% AcOH) as eluent (in gradient; flow 80 mL/min) to afford (II-trans) and (II-cis).

### Procedure B

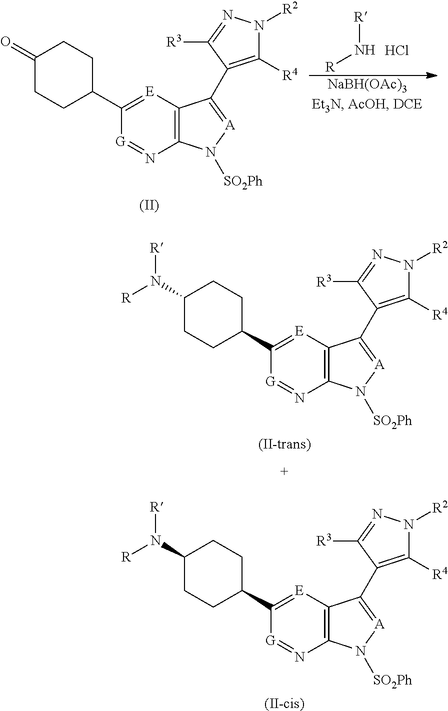

wherein A, E, G, R², R³ and R⁴ have the same meanings as described above, R and R′ are independently hydrogen or C₁₋₆alkyl, or R and R′, together with the nitrogen atom they are bonded to, form a 4-8 membered ring optionally substituted with halogen or C₁₋₆alkyl:
Et₃N (204 mg, 2.0 mmol) was added at RT to a mixture of secondary amine R′RNH hydrochloride (1.7 mmol) and ketone (II) (1 mmol) in dry 1,2-dichloroethane (7.1 mL), followed by glacial acetic acid (62 mg, 1.0 mmol) and NaBH(OAc)₃. When using free amine, Et₃N is omitted. The mixture was then stirred at RT overnight. Aqueous 10% NaOH (9 mL) was added, the mixture was stirred vigorously for 10 min and extracted with EtOAc (3×35 mL). The combined organic extracts were dried over MgSO₄ and concentrated. The crude product was purified by SGC) using a suitable solvent as eluent or by PTLC using appropriate solvent as the eluent or by LCMS (column LUNA 10μ C18(2) 00G-4253-V0 250×50 mm) using water-MeCN (0.1% AcOH) as eluent (in gradient; flow 80 mL/min) to afford (II-trans) and (II-cis).

### General Procedures for the Suzuki Reaction

### Procedure A

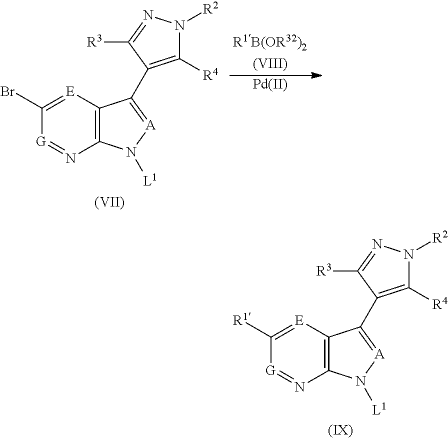

wherein A, E, G, R¹, R² and R³ have the same meanings as described above, R^{1′} is a suitable precursor to R¹ that can be transformed into R¹ by hydrogenation, and R³² is independently hydrogen or C₁₋₆alkyl or two R³² groups together form a five, six or seven membered optionally ring with the boron and oxygen atoms, the ring being optionally substituted with one or more C₁₋₆alkyl groups such as methyl or ethyl. Suitably, R³² is hydrogen or both R³² groups together form the group —C(CH₃)₂—C(CH₃)₂—. Bromide (VII) (1 mmol), boronic acid or boronic acid pinacol ester R^{1′}-B(OR³²)₂ (2 mmol), LiCl (3 mmol), and Pd(PPh₃)₂Cl₂ (0.1 mmol), were dissolved in EtOH (20 mL) and toluene (20 mL). Then 1.0 M Na₂CO₃ solution (20-25 mL) was added and the reaction was heated to 105-110° C. for 8 h. The reaction mixture was allowed to cool. It was poured into water (30 mL) and was extracted with EtOAc (3×40 mL). The combined organic extracts were dried over MgSO₄ and concentrated. Product (IX) was isolated by means of SGC using hexane/EtOAc as the eluent (gradient elution 0%-100% EtOAc) or by PTLC using a suitable solvent system as the eluent.

### Procedure B

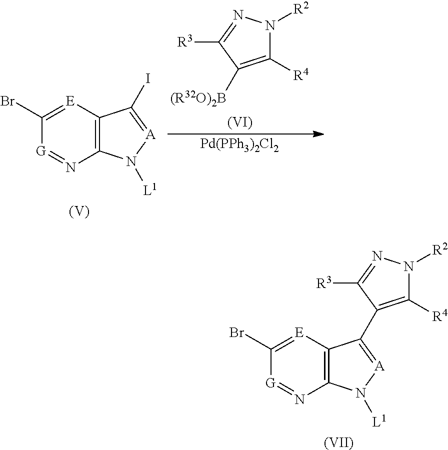

wherein A, E, G, R², R³, R⁴, L¹ and R³² have the same meanings as described above. A mixture of iodide (V) (10 mmol), boronic acid or boronic acid pinacol ester (VI) (11 mmol), LiCl (30 mmol), Pd(PPh₃)₂Cl₂ (0.5 mmol) and 1.0 M Na₂CO₃ solution (25 mL) in EtOH (25 mL) and toluene (25 mL) was heated at 100° C. for 3 h. The reaction mixture was cooled to RT, diluted with water (35 mL) and extracted with EtOAc (4×40 mL). The combined organic extracts were dried (MgSO₄) and concentrated. The product (VII) was isolated by crystallization and/or by SGC using a suitable solvent system as eluent. Yield 33-80%.

### General Procedure for the Synthesis of Enol Triflates

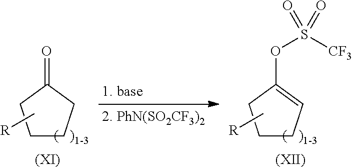

wherein R is the substituent on R¹. To a solution of ketone (XI) (10 mmol) in THF (35 mL), cooled to −78° C., was added 1.0 M solution of LiHMDS or NaHMDS in THF (12 mL, 12 mmol,) dropwise. The stirring continued at −78° C. for 1 h. N-phenylbis(trifluoromethanesulfonimide) (3.93 g, 11 mmol) was added in one portion and the stirring continued at −78° C. for 1 h then at RT for 19.5 h. The solvent was evaporated and the crude product purified by column chromatography on alumina (Neutral, Grade I) using hexane:EtOAc=7:1 (v/v) as the eluent. Alternatively, the product can be isolated by SGC using EtOAc:hexane:Et₃N=39:60:1 (v/v/v) as eluent (gradient elution starting with 19:80:1) to give triflate (XII). Yield 62-84%.

### General Procedure for the Synthesis of Boronic Pinacol Esters

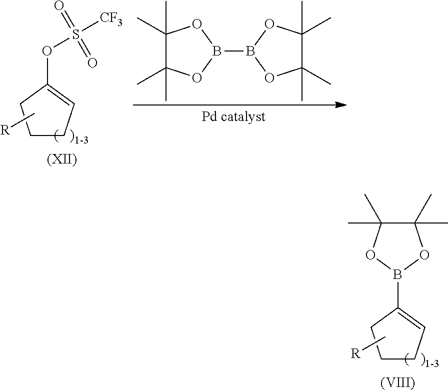

wherein R is the substituent on R¹. A mixture of triflate (XII) (10 mmol), bis(pinacolatodiboron) (3.80 g, 15 mmol), potassium acetate (2.94 g, 30 mmol) and dichloro[1,1′-bis(diphenylphosphino)ferrocene]palladium (II) dichloromethane adduct (0.41 g, 0.5 mmol) in DMF (43 mL) was stirred at 85° C. for 6-17 h to give a homogeneous black solution. The reaction mixture was concentrated and diluted with EtOAc. The solid was filtered off and the filtrate concentrated. The residue was purified by SGC using EtOAc:hexane=1:1 (v/v) as eluent (gradient elution) to give compound (VIII). Yield 47-67%.

### General Procedure for Protection of Diazaindoles as Phenylsulfonamides

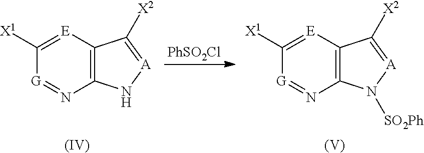

wherein A, E, G have the same meanings as described above, X¹ and X² are suitable halogen atoms such as bromine and iodine. Benzenesulfonyl chloride (15 mmol) was added to a stirred mixture of diazaindole (IV) (10 mmol), tetrabutylammonium hydrogensulfate (1.5 mmol) and 50% aqueous NaOH (4 mL) in CH₂Cl₂ (60 mL). The mixture was stirred at RT for 3.5 h while the progress of the reaction was followed by TLC. The mixture was then partitioned between CH₂Cl₂ (50 mL) and brine (80 mL). The aqueous layer was extracted with CH₂Cl₂ (3×60 mL) and the combined organic extracts dried (MgSO₄), filtered and concentrated. The residual semisolid was stirred with cold MeOH (70 mL) for 1.5 h. The resulting solid was filtered off and dried in vacuo to afford (V) in about 80% yield.

### Synthesis of Boronic Ester (VIII-6)

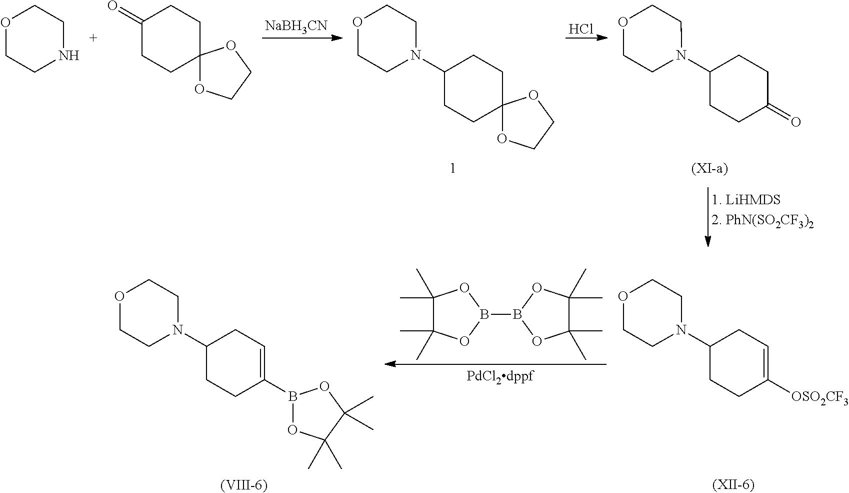

### 4-(1,4-Dioxa-spiro[4.5]dec-8-yl)-morpholine (1)

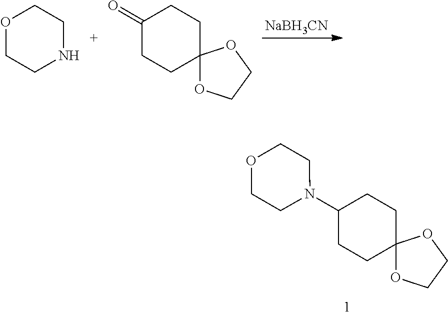

A mixture of 1,4-dioxaspiro[4.5]decan-8-one (10.0 g, 64.0 mmol), morpholine (20 mL) and AcOH (1.0 mL) was stirred for 2.5 h. Sodium cyanoborohydride (8.05 g, 128.0 mmol) was then added in one portion followed by more morpholine (15 mL). An exothermic reaction occurred and the mixture was cooled for 2 min with an ice-bath. Then the mixture was stirred at RT for 16 h. EtOH (120 mL) and water (28 mL) were added to the resulting thick slurry and the white solid filtered, washed with EtOH (2×) and the filtrate concentrated. EtOAc was then added, the precipitate filtered off, washed with EtOAc and the filtrate concentrated. The residual oil was purified by Kugelrohr distillation to give compound 1 (9.03 g, 62%; b.p. 140° C./0.05 mmHg) as a clear oil which solidified on standing. ¹H NMR (400 MHz; CDCl₃) δ 1.51-1.68 (m, 4H), 1.81-1.84 (m, 4H), 2.28-2.34 (m, 1H), 2.57 (t, J 4.7, 4H), 3.72 (t, J 4.7, 4H), 3.95 (s, 4H).

### 4-(1,4-Dioxa-spiro[4.5]dec-8-yl)-morpholine (1)

### An Alternative Method

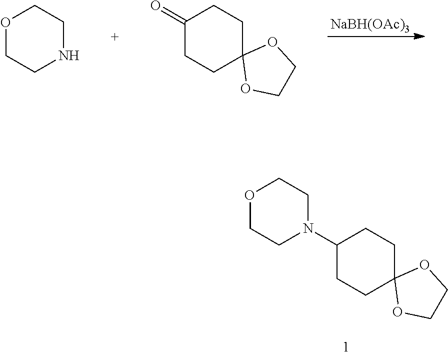

Sodium triacetoxyborohydride (382 g, 1.8 mol) was added in one portion to a mixture of 1,4-dioxaspiro[4.5]decan-8-one (200.0 g, 1.28 mol), morpholine (111.4 g, 1.28 mol) and glacial AcOH (73.2 mL, 1.28 mol) in 1,2-dichloroethane (4 L). A slightly exothermic reaction occurred accompanied by increase in temperature by 12° C. Then the mixture was stirred at RT overnight. The reaction was quenched by the addition of 10% aqueous NaOH (1.8 L) over a period of 20 min. The organic layer was separated, washed with brine (1 L), dried over MgSO₄ and concentrated to afford compound 1 (237.66 g) as white solid. The aqueous layer was extracted with EtOAc (4×300 mL). Combined extracts were washed with brine (1 L), dried over MgSO₄ and concentrated to furnish an additional portion of compound 1 (44 g) as an off-white solid. Total yield of compound 1 (281.66 g, 97%). ¹H NMR data identical with the data obtained for first method.

### 4-Morpholin-4-yl-cyclohexanone (XI-a)

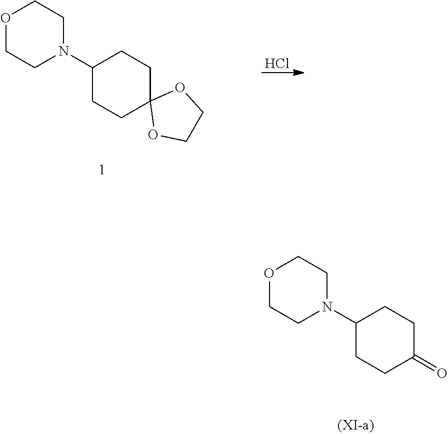

To a solution of compound 1 (4.50 g, 19.8 mmol) in THF (100 mL) was added 7 N aqueous HCl (40 mL). The reaction mixture was stirred for 17 h and the reaction was quenched by pouring onto saturated aqueous NaHCO₃ (475 mL). The mixture was extracted with EtOAc (1×) then CH₂Cl₂ (3×) and the combined organic extracts dried (MgSO₄) and concentrated. The resulting oil was purified by Kugelrohr distillation to give (XI-a) (3.17 g, 87%) as a clear oil; ¹H NMR (400 MHz, CDCl₃) δ1.80-1.94 (m, 2H), 1.80-2.10 (m, 2H), 2.30 (m, 1H), 2.45-2.65 (m, 8H), 3.74 (t, J 4.7, 4H).

### Trifluoromethanesulfonic acid 4-morpholin-4-yl-cyclohex-1-enyl ester (XII-6)

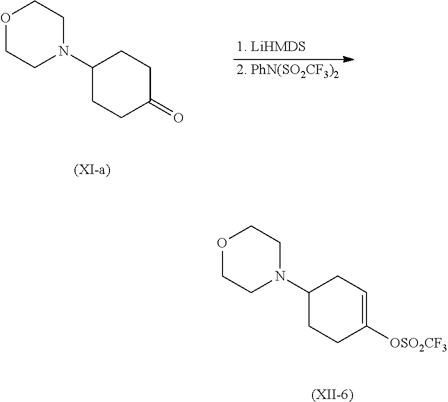

Triflate (XII-6) was prepared using the general procedure for the synthesis of enol triflates using ketone (XI-a) (5.30 g, 28.9 mmol), 1 M solution of LiHMDS in THF (34.7 mL, 34.7 mmol) and N-phenylbis(trifluoromethanesulfinimide) (11.37 g, 31.8 mmol) in dry THF (100 mL). The crude product was purified by SGC using EtOAc:hexane:Et₃N=39:60:1 (v/v/v) as eluent (gradient elution starting with 19:80:1) to give triflate (XII-6) (7.66 g, 84%) as an orange oil; ¹H NMR (400 MHz, CDCl₃) δ 1.61-1.72 (m, 1H), 2.07 (m, 1H), 2.20 (m, 1H), 2.30-2.48 (m, 3H), 2.50-2.65 (m, 5H), 3.72 (t, J 4.7, 4H), 5.72 (m, 1H).

### 4-[4-(4,4,5,5-Tetramethyl-[1,3,2]dioxazolidin-2-yl)-cyclohex-3-enyl]-morpholine (VIII-6)

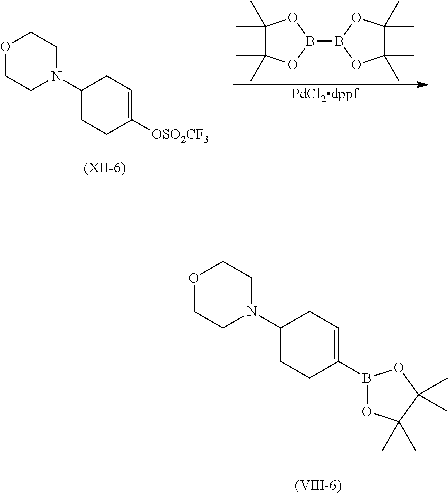

The compound was prepared using the general procedure for the synthesis of boronic pinacol esters. Triflate (XII-6) (8.00 g, 25.4 mmol), bis(pinacolatodiboron) (9.66 g, 38.1 mmol), potassium acetate (7.47 g, 76.1 mmol) and dichloro[1,1′-bis(diphenylphosphino)ferrocene]palladium (II) dichloromethane adduct (1.04 g, 1.27 mmol) in DMF (110 mL) was stirred at 85° C. for 17 h. The crude product was purified by SGC using EtOAc:hexane=1:1 (v/v) as eluent (gradient elution) to give (VIII-6) (4.95 g, 67%) as a light orange solid; ¹H NMR (400 MHz, CDCl₃) δ 1.26 (s, 12H), 1.95-2.10 (m, 2H), 2.05-2.20 (m, 2H), 2.80-2.40 (m, 2H), 2.43-2.65 (m, 5H), 3.74 (t, J 4.7, 4H), 6.51 (m, 1H).

### Synthesis of Boronic Ester (VIII-3)

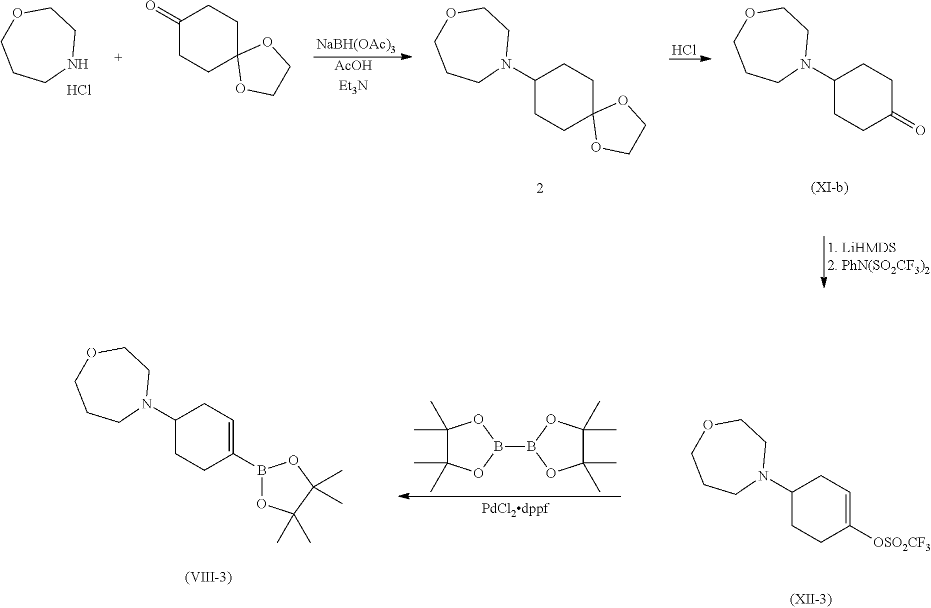

### 4-(1,4-dioxaspiro[4.5]decan-8-yl)-1,4-oxazepane (2)

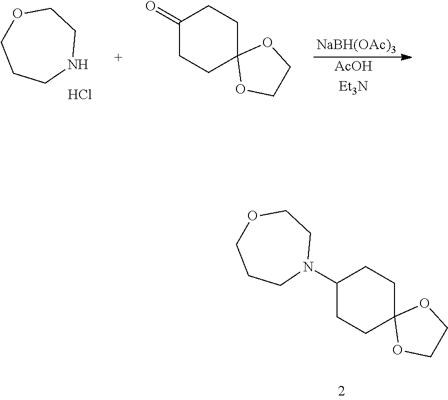

Et₃N (96.9 mL, 0.695 mol) was added in one portion to a stirred suspension of homomorpholine hydrochloride (79.76 g, 0.579 mol) and 1,4-dioxaspiro[4.5]decan-8-one (90.5 g, 0.579 mol) in 1,2-dichloroethane (1.81 L). Then glacial acetic acid (34.8 mL, 0.607 mol) was added in one portion followed by solid NaBH(OAc)₃ (154 g, 0.727 mol) in one portion as well. This was accompanied by a 5° C. increase in the temperature of the reaction mixture. After 2 h 45 min, the reaction was quenched by addition of 10% aqueous NaOH (800 mL). The mixture was stirred for 10 min. The organic layer was separated, washed with brine (100 mL), dried (MgSO₄) and concentrated to afford an oil (142.36 g) with some suspended solid, which was filtered off (3.00 g). The aqueous part of the reaction mixture was combined with the brine washings and extracted with EtOAc (4×500 mL). Combined extracts were washed with brine (100 mL), dried (MgSO₄) and concentrated to afford additional portion of oil (16.82 g). The two oily products were combined and distilled in vacuo to give oxazepane 2 (101.07 g, 72%) as colorless liquid, b.p. 122° C./8.9·10⁻³ mbar. ¹H NMR (400 MHz, CDCl₃) δ 1.48-1.64 (m, 4H), 1.70-1.88 (m, 6H), 2.50-2.63 (m, 1H), 2.71-2.81 (m, 4H), 3.66-3.72 (m, 2H), 3.78 (t, J 6.0, 2H), 3.93 (s, 4H).

### 4-(1,4-oxazepan-4-yl)cyclohexanone (XI-b)

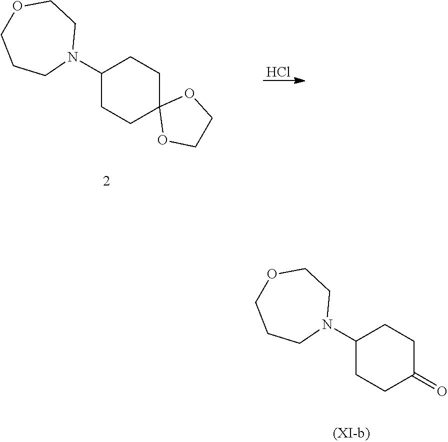

To a cooled (<15° C.) solution of oxazepane 2 (10.31 g, 42.75 mmol) in THF (216 mL) was added 7 N aqueous HCl (86 mL, 0.602 mol) over a period of 5 min. The cooling bath was then removed and the reaction mixture was stirred overnight at RT. Then, the reaction mixture was basified to pH 8 by dropwise addition of 50% aqueous NaOH (48 g, 0.602 mol) over a period of 30 min while maintaining the internal temperature at 10-13° C. using an external cooling bath (0° C.). Hexane (50 mL) was added and the organic layer was separated, dried over MgSO₄ and concentrated to afford a yellowish liquid (7.21 g). The aqueous layer was extracted with EtOAc (4×50 mL). The extracts were combined, dried (MgSO₄) and concentrated to afford the second portion of crude product (1.58 g). Both portions of crude product were combined and distilled in vacuo to afford ketone (XI-b) (7.27 g, 86%) as colorless liquid, b.p. 98° C./5.3·10⁻³ mbar; ¹H NMR (400 MHz, CDCl₃) δ 1.73-1.85 (m, 2H), 1.89 (quintet, J 5.9, 2H), 2.05-2.15 (m, 2H), 2.30-2.42 (m, 2H), 2.43-2.52 (m, 2H), 2.79-2.85 (m, 4H), 3.03 (tt, J 10.4, 6.6, 1H), 3.72-3.77 (m, 2H), 3.82 (t, J 6.0, 2H).

### 4-(1,4-oxazepan-4-yl)cyclohex-1-enyl trifluoromethanesulfonate (XII-3)

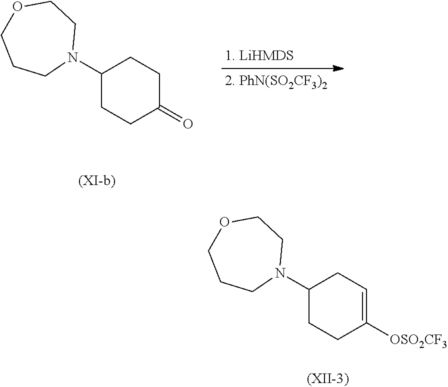

Triflate (XII-3) was prepared using the general procedure for the synthesis of enol triflates using ketone (XI-b) (6.49 g, 32.9 mmol), 1 M solution of LiHMDS in THF (39.5 mL, 39.5 mmol) and N-phenylbis(trifluoromethanesulfinimide) (12.94 g, 36.2 mmol) in dry THF (115 mL). The crude reaction mixture was diluted with hexane:EtOAc=4:1 (115 mL) (v/v) and washed with water (50 mL), brine (50 mL), dried (MgSO₄) and concentrated. The liquid residue was distilled in vacuo to afford (XII-3) (6.98 g, 64%) as colorless liquid b.p 114° C./5.7·10⁻³ mbar. Purity about 85% by ¹H NMR. ¹H NMR (400 MHz, CDCl₃) δ 1.63-1.76 (m, 1H), 1.86 (quintet, J 6.0, 2H), 1.95-2.05 (m, 1H), 2.12-2.24 (m, 1H), 2.26-2.56 (m, 3H), 2.74-2.80 (m, 4H), 2.82-2.92 (m, 1H), 3.68-3.74 (m, 2H), 3.79 (t, J 6.0, 2H), 5.72 (dt, J 5.7, 2.4, 1H).

### 4-(4-(4,4,5,5-tetramethyl-1,3,2-dioxaborolan-2-yl)cyclohex-3-enyl)-1,4-oxazepane (VIII-3)

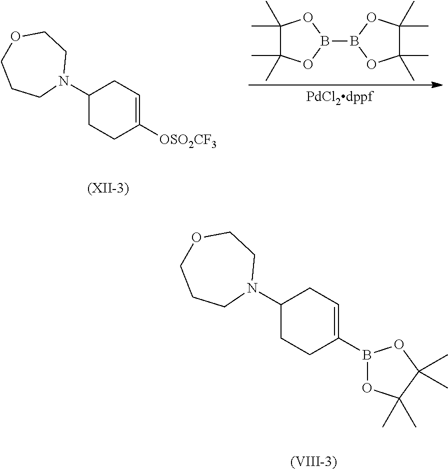

The compound was prepared using the general procedure for the synthesis of boronic pinacol esters. Triflate (XII-3) (6.60 g, 20.06 mmol), bis(pinacolatodiboron) (7.62 g, 30.09 mmol), AcOK (5.90 g, 60.2 mmol) and dichloro[1,1′-bis(diphenylphosphino)ferrocene]palladium (II) dichloromethane adduct (0.82 g, 1.0 mmol) in DMF (86 mL) was stirred at 85° C. for 1 h 45 min when TLC showed absence of the remaining starting material. The mixture was concentrated and separated between EtOAc (125 mL)-water (125 mL). The organic layer was washed with water (120 mL), dried (MgSO₄), concentrated and separated by means of chromatography on amino silica (Chromatorex NH, Fuji Silysia) using hexane-EtOAc as eluent (gradient elution) to afford (VIII-3) (2.149 g, 35%) as white solid; ¹H NMR (400 MHz, CDCl₃) δ 1.26 (s, 12H), 1.33-1.47 (m, 1H), 1.83-1.93 (m, 3H), 2.04-2.22 (m, 2H), 2.23-2.38 (m, 2H), 2.75-2.84 (m, 5H), 3.71 (t, J 4.7, 2H), 3.80 (t, J 6.0, 2H), 6.52 (m, 1H).

### EXAMPLE 1

### 5-cyclohexyl-3-(1-methyl-1H-pyrazol-4-yl)-1H-pyrazolo[3,4-b]pyridine (I-A-2)

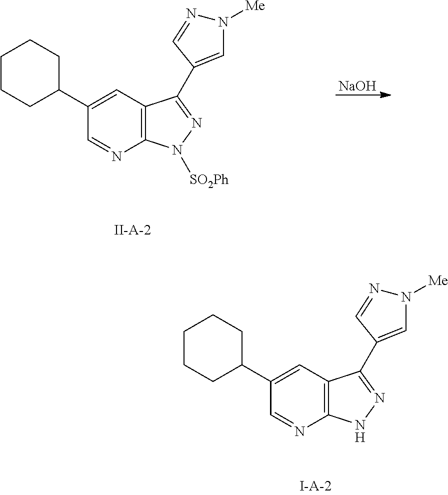

Compound II-A-2 (63.5 mg, 0.15 mmol) was heated at 90° C. for 1 h in a mixture of 10% aqueous NaOH (0.7 mL): EtOH (8 mL). The reaction mixture was then cooled to RT, diluted with EtOAc (20 mL) and washed with saturated NaHCO₃ solution (3×15 mL). The organic layer was dried over MgSO₄ and concentrated to afford I-A-2 (34 mg, 80%). ¹H NMR (400 MHz, CDCl₃) δ 1.41-1.59 (m, 5H), 1.78-2.01 (m, 5H), 2.67-2.77 (m, 1H), 4.03 (s, 3H), 7.93 (s, 1H), 8.01 (d, J 2.0, 1H), 8.05 (s, 1H), 8.52 (d, J 2.0, 1H), 11.62 (brs, NH).

### EXAMPLE 2

### 4-((1r,4r)-4-(3-(1-methyl-1H-pyrazol-4-yl)-1H-pyrazolo[3,4-b]pyridin-5-yl)cyclohexyl)-1,4-oxazepane (I-A-4)

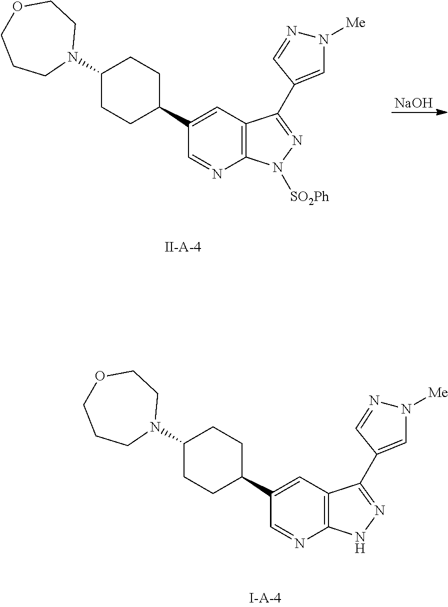

A mixture of azaindazole II-A-4 (36.6 mg, 7.03 mmol), 10% NaOH solution (0.5 mL) and EtOH (5 mL) was heated at 100° C. for 1 h. Then it was cooled to RT, diluted with EtOAc (20 mL), washed with saturated NaHCO₃ solution (3×15 mL), dried (MgSO₄) and concentrated. The residue (20.5 mg) was purified by PTLC using CHCl₃:MeOH:NH₄OH=93:6:1 (v/v/v) as eluent to afford azaindazole I-A-4 (5.5 mg, 21%). ¹H NMR (400 MHz, CDCl₃) δ 1.50-1.75 (m, 6H), 1.89-2.25 (m, 4H), 2.66-2.77 (m, 2H), 2.83-3.09 (m, 4H), 3.84 (t, J 6.2, 4H), 4.03 (s, 3H), 7.92 (s, 1H), 7.99 (d, J 2.0, 1H), 8.03 (s, 1H), 8.48 (d, J 2.0, 1H) and 11.15 (brs, NH). MS (ES) m/z 381 (MH⁺).

### EXAMPLE 3

### 4-((1s,4s)-4-(3-(1-methyl-1H-pyrazol-4-yl)-1H-pyrazolo[3,4-b]pyridin-5-yl)cyclohexyl)-1,4-oxazepane (I-A-5)

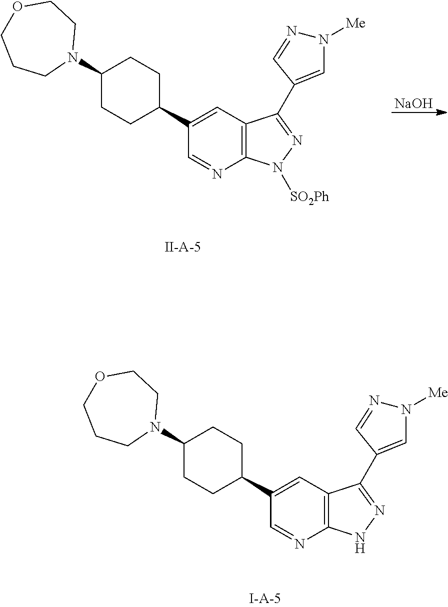

A mixture of azaindazole II-A-5 (18.5 mg, 0.03 mmol), 10% NaOH solution (0.5 mL) and EtOH (5 mL) was heated at 100° C. for 1 h. Then it was cooled to RT, diluted with EtOAc (20 mL), washed with saturated NaHCO₃ solution (3×15 mL), dried (MgSO₄) and concentrated. The residue (11 mg) was purified by PTLC using CHCl₃:MeOH:NH₄OH=93:6:1 (v/v/v) as eluent to afford azaindazole I-A-5 (3.5 mg, 26%). MS (ES) m/z 381 (MH⁺).

### EXAMPLE 4

### 2-cyclohexyl-7-(1-methyl-1H-pyrazol-4-yl)-5H-pyrrolo[3,2-b]pyrazine (I-E-2)

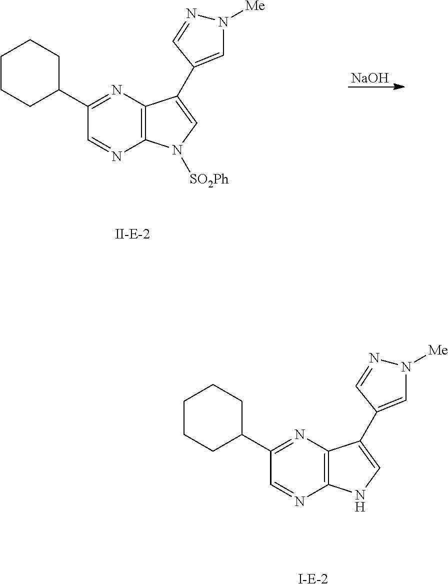

Compound II-E-2 (68 mg, 0.1613 mmol) was heated at 80° C. for 20 min in a mixture of 10% aqueous NaOH (1 mL): EtOH (2 mL). The reaction mixture was then cooled to RT, diluted with a saturated solution of NaHCO₃ (15 mL) and extracted with EtOAc (3×15 mL). The combined organic extracts were dried over MgSO₄, concentrated and purified by PTLC using EtOAc as eluent to afford I-E-2 (30 mg, 66%) as a yellow solid. ¹H NMR (400 MHz, CDCl₃) δ 1.29-1.42 (m, 1H), 1.49 (qt, J 12.72, 3.28, 2H), 1.72 (qd, J 12.46, 3.28, 2H), 1.78-1.86 (m, 1H), 1.93 (dt, J 13.01, 3.03, 2H), 2.05 (dd, J 13.64, 1.89, 2H), 2.91 (tt, J 11.91, 3.51, 1H), 4.02 (s, 3H), 7.68 (d, J 2.78, 1H), 7.95 (d, J 0.63, 1H), 8.18 (s, 1H), 8.21 (s, 1H), 9.31 (br. s, 1H).

### 5-cyclohexyl-3-(1-methyl-1H-pyrazol-4-yl)-1-(phenylsulfonyl)-1H-pyrazolo[3,4-b]pyridine (II-A-2)

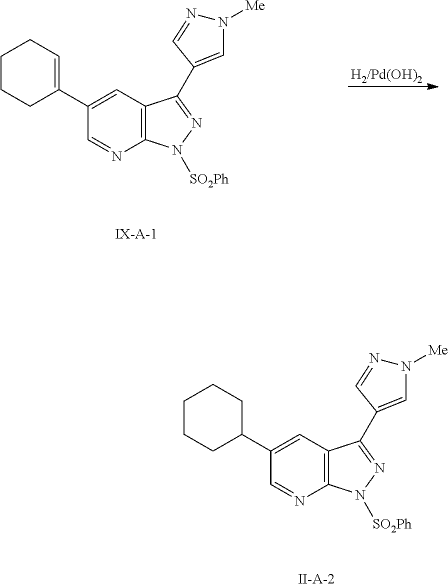

Compound IX-A-1 (0.115 g, 0.275 mmol) and Pd(OH)₂ (0.155 g) in EtOAc:MeOH=1:1 (10 mL; v/v) was stirred vigorously under the H₂ for 20.5 h. The catalyst was filtered off on Celite (washing with MeOH:EtOAc=4:6, 500 mL, v/v), the filtrate was concentrated and the residual oil purified by PTLC using CH₂Cl₂:MeOH=98:2 (v/v) as eluent to afford azaindazole II-A-2 (63.8 mg, 55%). ¹H NMR (400 MHz, CDCl₃) δ 1.37-1.53 (m, 4H), 1.62-1.70 (m, 1H), 1.75-1.83 (m, 1H), 1.84-1.97 (m, 4H), 2.65-2.75 (m, 1H), 4.00 (s, 3H), 7.46 (t, J 7.6, 2H), 7.56 (t, J 7.6, 1H), 7.91 (s, 1H), 7.99-8.04 (m, 2H), 8.15-8.20 (m, 2H), 8.61 (d, J 2.1, 1H).

### 4-((1r,4r)-4-(3-(1-methyl-1H-pyrazol-4-yl)-1-(phenylsulfonyl)-1H-pyrazolo[3,4-b]pyridin-5-yl)cyclohexyl)-1,4-oxazepane (II-A-4) and 4-((1s,4s)-4-(3-(1-methyl-1H-pyrazol-4-yl)-1-(phenylsulfonyl)-1H-pyrazolo[3,4-b]pyridin-5-yl)cyclohexyl)-1,4-oxazepane (II-A-5)

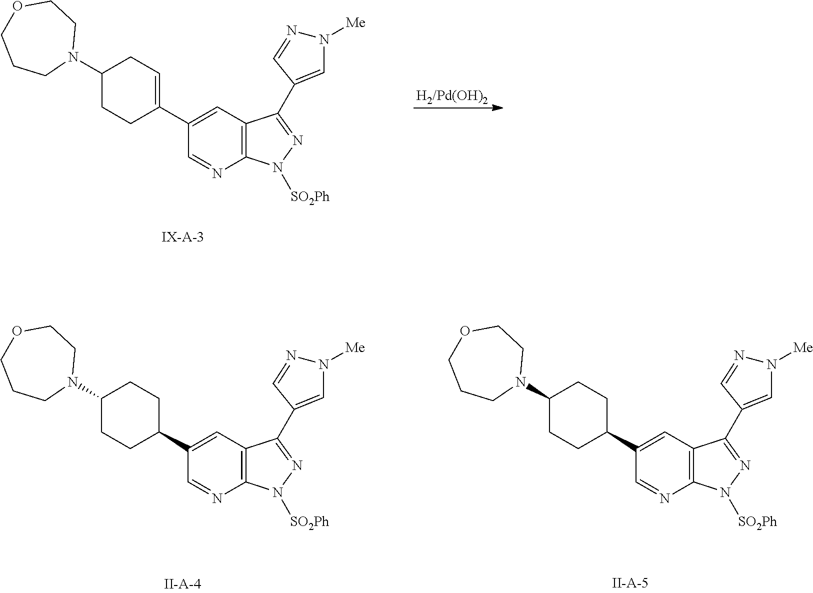

A solution of the azaindazole IX-A-3 (0.143 g, 0.275 mmol) in MeOH (5 mL) and EtOAc (5 mL) was stirred vigorously for 24 h with Pd(OH)₂ (0.166 g; wet Degussa type on carbon) under H₂. The mixture was then filtered through Celite with washing with MeOH (200 mL) and EtOAc (300 mL), and the combined filtrates concentrated to afford mainly the azaindazole IX-A-3 and small amount of II-A-4 and II-A-5. This mixture was redissolved in MeOH (5 mL) and EtOAc (5 mL) and stirred again for 24 h with Pd(OH)₂ (0.18 g; wet Degussa type on carbon) under H₂. The mixture was filtered through Celite and washed with MeOH-EtOAc as before and concentrated. The resulting oil was purified by PTLC using CHCl₃:MeOH:NH₄OH=93:6:1 (v/v/v) as eluent to afford trans isomer II-A-4 (36.6 mg, 26%) and cis isomer II-A-5 (18.5 mg, 13%).

Data for the trans isomer II-A-4: ¹H NMR (400 MHz, CDCl₃) δ 1.49-1.69 (m, 6H), 1.90-2.22 (m, 4H), 2.66-2.76 (m, 2H), 2.84-3.07 (m, 4H), 3.82 (t, J 6.2, 4H), 4.00 (s, 3H), 7.44-7.50 (m, 2H), 7.56 (tt, J 7.4, 1.5, 1H), 7.91 (d, J 2.1, 1H), 8.02 (s, 2H), 8.15-8.20 (m, 2H), 8.60 (d, J 2.1, 1H).

### EXAMPLE 5

### 4-((1r,4r)-4-(7-(1-methyl-1H-pyrazol-4-yl)-5H-pyrrolo[3,2-b]pyrazin-2-yl)cyclohexyl)morpholine (I-E-7)

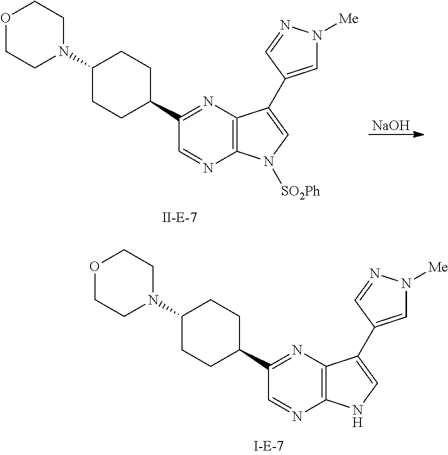

Compound II-E-7 (545 mg, 1.0757 mmol) was heated at 80° C. for 25 min in a mixture of 10% aqueous NaOH (4.3 mL): EtOH (10 mL). The reaction mixture was then cooled to RT, diluted with a saturated solution of NaHCO₃ (50 mL) and extracted with EtOAc (4×50 mL). The combined organic extracts were dried over MgSO₄, concentrated and purified by preparative TLC (PTLC) using CH₂Cl₂:MeOH=9:1 as eluent to afford I-E-7 (213 mg, 54%) as a pale yellow solid. ¹H NMR (400 MHz, CDCl₃) δ 1.37-1.59 (m, 2H), 1.73-1.87 (m, 2H), 2.15 (d, J 10.2, 4H), 2.42 (t, J 11.7, 1H), 2.67 (t, J 4.2, 4H), 2.88 (tt, J 12.1, 3.2, 1H), 3.79 (t, J 4.4, 4H), 4.01 (s, 3H), 7.67 (d, J 2.8, 1H), 7.96 (d, J 0.6, 1H), 8.17 (s, 1H), 8.17 (s, 1H), 9.09 (br s, 1H). MS (ES) MH⁺m/e=367.2.

### 2-cyclohexyl-7-(1-methyl-1H-pyrazol-4-yl)-5-(phenylsulfonyl)-5H-pyrrolo[3,2-b]pyrazine (II-E-2)

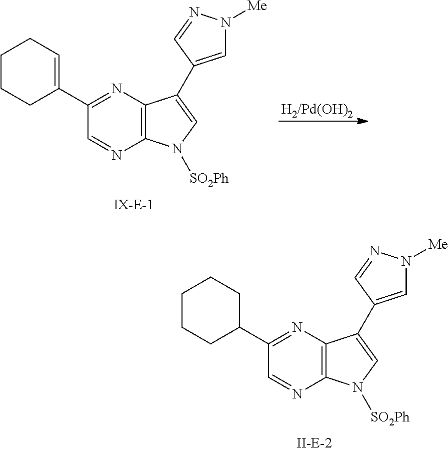

Compound IX-E-1 (100 mg, 0.238 mmol) and Pd(OH)₂ (10.04 mg, 0.072 mmol) in EtOAc:MeOH=1:1 (20 mL; v/v) was stirred vigorously under H₂ overnight. To reduce the starting material still present in the reaction mixture the catalyst was filtered off on Celite (washing with CH₂Cl₂:MeOH=9:1, 100 mL, v/v), the filtrate was concentrated and the residue hydrogenated again as described above. The crude product II-E-2 (68 mg, 67%) was isolated as a white foam, which did not require further purification. ¹H NMR (400 MHz, CDCl₃) δ 1.22-1.38 (m, 2H), 1.39-1.54 (m, 2H), 1.64 (qd, J 12.34, 2.53, 2H), 1.73-2.03 (m, 4H), 2.87 (tt, J 11.78, 3.51, 1H), 4.01 (s, 3H), 7.48-7.54 (m, 2H), 7.57-7.63 (m, 1H), 8.00 (s, 1H), 8.03 (s, 1H), 8.15-8.20 (m, 3H), 8.27 (s, 1H).

### 4-((1r,4r)-4-(7-(1-methyl-1H-pyrazol-4-yl)-5-(phenylsulfonyl)-5H-pyrrolo[3,2-b]pyrazin-2-yl)cyclohexyl)morpholine (II-E-7) and 4-((1s,4s)-4-(7-(1-methyl-1H-pyrazol-4-yl)-5-(phenylsulfonyl)-5H-pyrrolo[3,2-b]pyrazin-2-yl)cyclohexyl)morpholine (II-E-8)

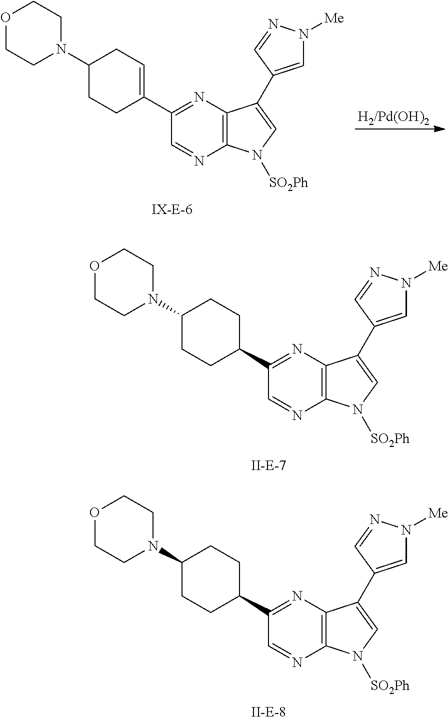

Compound IX-E-6 (1.2 g, 2.3781 mmol) and 20% Pd(OH)₂ (0.835 g wet Degussa type; 0.167 g of Pd(OH)₂, 1.189 mmol) in EtOAc:MeOH=1:1 (47.5 mL; v/v) was stirred vigorously under H₂ overnight. The catalyst was filtered off on Celite (washing with CH₂Cl₂:MeOH=1:1, 500 mL, v/v), the filtrate was concentrated and the residual white foam was separated by SGC using EtOAc:MeOH (gradient elution from 98:2 to 85:15, v/v) followed by CH₂Cl₂:MeOH=9:1 to give the cis isomer II-E-8 as a white solid (201 mg, 17%) and the trans isomer II-E-7 as a white foam (545 mg, 45%).

Data for the trans isomer II-E-7: ¹H NMR (400 MHz, CDCl₃) δ 1.47 (dq, J 4.42, 3.03, 2H), 1.73 (dq, J 4.17, 2.91, 2H), 2.10 (t, J 11.24, 4H), 2.32-2.41 (m, 1H), 2.63 (m, J 4.55, 4.55, 4H), 2.84 (tt, J 12.05, 3.36, 1H), 3.76 (m, J 4.55, 4.55, 4H), 4.00 (s, 3H), 7.47-7.54 (m, 2H), 7.57-7.63 (m, 1H), 8.02 (d, J 0.76, 1H), 8.03 (s, 1H), 8.14 (s, 1H), 8.17-8.20 (m, 2H), 8.27 (s, 1H). MS (ES) MH⁺ m/e=507.2.

Data for the cis isomer II-E-8: ¹H NMR (400 MHz, CDCl₃) δ 1.60-1.69 (m, 2H), 1.75 (dq, J 16.3, 3.7, 2H), 1.86-1.97 (m, 2H), 2.15-2.27 (m, 2H), 2.31 (dt, J 5.6, 3.3, 1H), 2.50 (br s, 4H), 3.08 (tt, J 8.7, 4.3, 1H), 3.73 (t, J 4.7, 4H), 4.00 (s, 3H), 7.48-7.53 (m, 2H), 7.57-7.62 (m, 1H), 8.02 (d, J 0.6, 1H), 8.04 (s, 1H), 8.16-8.20 (m, 3H), 8.34 (s, 1H). MS (ES) MH⁺ m/e=507.2.

### 5-bromo-3-iodo-1H-pyrazolo[3,4-b]pyridine (IV-A-1)

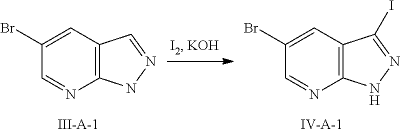

To a stirred solution of the bromo-diazaindole III-A-1 (1 g, 5.05 mmol) in DMF (14 mL) was added crushed KOH pellets (1.06 g, 19.03 mmol) in one portion. After 11 min, I₂ (1.15 g, 4.54 mmol) was added and the mixture stirred vigorously for 3.5 h. The mixture was then partially concentrated in vacuo, diluted with EtOAc (40 mL):saturated NaHCO₃ solution (20 mL) and partitioned. The aqueous layer was extracted with EtOAc (2×20 mL). The combined organic layers were dried (MgSO₄), filtered and concentrated to afford a 1:1 mixture of III-A-1 and IV-A-1 (1.377 g). This mixture was re-dissolved in 1,4-dioxane (14 mL) and treated with solid NaOH (0.7 g). The mixture was stirred for 5 min at RT and I₂ (0.7 g) was added. The mixture was stirred at 40° C. for 23 h, diluted with EtOAc (50 mL) and washed with saturated aqueous Na₂S₂O₃ solution (30 mL). The aqueous layer was extracted with EtOAc (2×30 mL) and the combined organic extracts were dried (MgSO₄), filtered and concentrated to afford azaindazole IV-A-1 (1.585 g, 97%). ¹H NMR (400 MHz; CDCl₃) δ 7.94 (d, J 2.1, 1H), 8.55 (d, J 2.1, 1H), 10.85 (brs, NH).

### 2-bromo-7-iodo-5H-pyrrolo[3,2-b]pyrazine (IV-E-1)

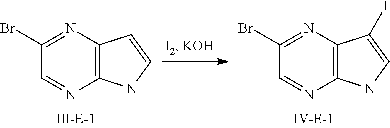

A mixture of III-E-1 (5.0 g, 25 mmol; commercially available from Ark pharma) and freshly ground KOH (5.10 g, 90.9 mmol) in DMF (100 mL) was stirred at RT under N₂ for 30 min. Iodine (6.35 g, 25.02 mmol) was then added in one portion and the red mixture was stirred at RT for 2 h when TLC indicated that the reaction was complete. The mixture was poured into water (100 mL) and extracted with EtOAc (3×100 mL). The combined organic extracts were dried and concentrated to give IV-E-1 (7.74 g, 95%) as a brown solid. ¹H NMR (400 MHz, CDCl₃) δ 7.67 (s, 1H), 8.23 (s, 1H).

### tert-butyl 5-bromo-3-iodo-1H-pyrazolo[3,4-b]pyridine-1-carboxylate (V-A-1)

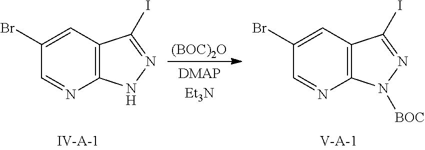

To a stirred solution of the azaindazole IV-A-1 (0.5 g, 1.54 mmol) in THF (10 mL) was added (BOC)₂O (0.354 g, 1.62 mmol), Et₃N (0.22 mL, 1.54 mmol) and a catalytic amount of DMAP. The mixture was stirred for 3 h, diluted with EtOAc (30 mL) and saturated NaHCO₃ solution (15 mL), and partitioned. The aqueous layer was extracted with EtOAc (2×20 mL). The combined organic layers were dried (MgSO₄) and concentrated to afford the protected azaindazole V-A-1 (0.611 g, 93%) as a tan solid. ¹H NMR (400 MHz; CDCl₃) δ 1.72 (s, 9H), 7.98 (d, J 2.1, 1H), 8.78 (d, J 2.1, 1H).

### 2-bromo-7-iodo-5-(phenylsulfonyl)-5H-pyrrolo[3,2-b]pyrazine (V-E-1)

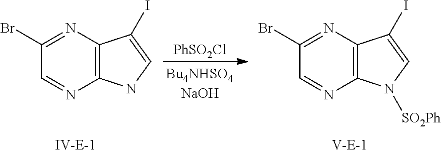

A mixture of (IV-E-1) (7.74 g, 23.9 mmol), PhSO₂Cl (6.54 g, 4.73 mL, 37.04 mmol), Bu₄NHSO₄ (1.217 g, 3.584 mmol) and 50% aqueous NaOH (5 mL, 7.65 g, 95.6 mmol) in CH₂Cl₂ (100 mL) was stirred vigorously at RT for 3 h. A saturated solution of NaHCO₃ (50 mL) was added and the mixture was extracted with CH₂Cl₂ (4×50 mL). The combined organic extracts were dried (MgSO₄), concentrated, and the residue was triturated with MeOH (100 mL). The solid was filtered off and dried in vacuo to afford V-E-1 (9.34 g, 85%) as yellow solid. ¹H NMR (400 MHz, CDCl₃) δ 7.49-7.60 (m, 2H), 7.62-7.73 (m, 1H), 8.15 (d, J 0.51, 1H), 8.16-8.21 (m, 2H), 8.44 (d, J 0.38, 1H). MS (ES) M⁺ m/e=464.

### 5-bromo-3-(1-methyl-1H-pyrazol-4-yl)-1-(phenylsulfonyl)-1H-pyrazolo[3,4-b]pyridine (VII-A-1)

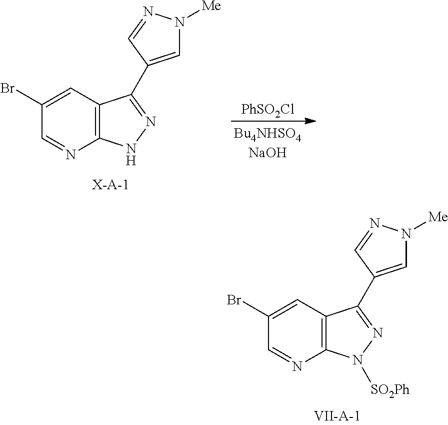

A mixture of (X-A-1) (0.394 g, 1.42 mmol), PhSO₂Cl (0.28 mL, 2.20 mmol), Bu₄NHSO₄ (0.072 g, 0.21 mmol) and 50% aqueous NaOH (1.9 mL, 2.91 g, 36.3 mmol) in CH₂Cl₂ (40 mL) was stirred vigorously at RT for 2 h. After 2 h the mixture was diluted with CH₂Cl₂ (40 mL) and brine (20 mL) and partitioned. The aqueous layer was extracted with CH₂Cl₂ (3×30 mL) and the combined organic extracts dried (MgSO₄), filtered and concentrated. The residual orange solid was stirred in cold MeOH (50 mL) for 10 min. The resulting solid was filtered off to afford azaindazole VII-A-1 as a tan powder (0.242 g, 41%). ¹H NMR (400 MHz, CDCl₃) δ 4.01 (s, 3H), 7.46-7.52 (m, 2H), 7.59 (tt, J 7.6, 1.4, 1H), 8.01 (s, 2H), 8.15-8.19 (m, 2H), 8.28 (d, J 2.1, 1H) and 8.75 (d, J 2.1, 1H).

### 2-bromo-7-(1-methyl-1H-pyrazol-4-yl)-5-(phenylsulfonyl)-5H-pyrrolo[3,2-b]pyrazine (VII-E-1)

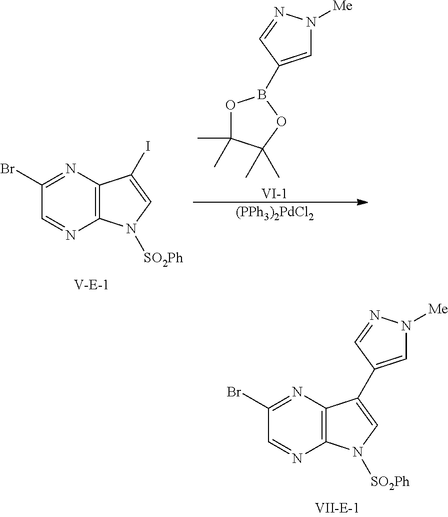

A mixture of V-E-1 (1.1 g, 2.37 mmol), boronic ester VI-1 (0.5425 g, 2.607 mmol), LiCl (0.2512 g, 5.926 mmol), 1.0 M Na₂CO₃ (5.93 mL, 5.93 mmol) and (PPh₃)₂PdCl₂ (0.1664 g, 0.237 mmol) in EtOH:toluene=1:1 (v/v) (47.4 mL) was heated at 110° C. with stirring for 4 h. The mixture was cooled to RT. Water (100 mL) was added and the mixture was extracted with EtOAc (3×100 mL). The combined organic extracts were dried over MgSO₄, concentrated and purified by SGC using hexane:EtOAc in gradient (100% hexane to 100% EtOAc) to give VII-E-1 (0.33 g, 33%) as a yellow solid. ¹H NMR (400 MHz, CDCl₃) δ 3.99 (s, 3H), 7.50-7.56 (m, 2H), 7.60-7.68 (m, 1H), 7.92 (s, 1H), 8.10 (s, 1H), 8.16 (s, 1H), 8.16-8.19 (m, 2H), 8.46 (d, 1H).

### 5-cyclohexenyl-3-(1-methyl-1H-pyrazol-4-yl)-1-(phenylsulfonyl)-1H-pyrazolo[3,4-b]pyridine (IX-A-1)

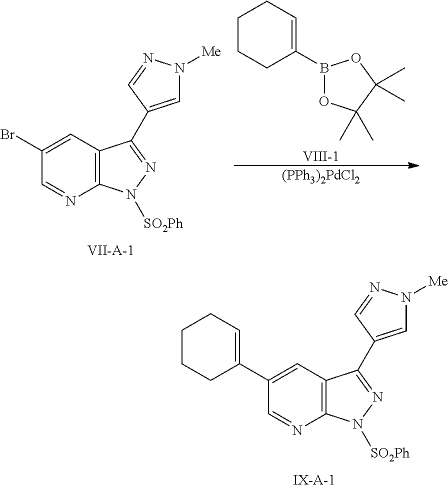

A mixture of VII-A-1 (0.115 g, 0.275 mmol), boronic ester VIII-1 (0.069 g, 0.33 mmol), LiCl (0.035 g, 0.825 mmol), 1.0 M Na₂CO₃ (0.74 mL, 0.742 mmol) and (PPh₃)₂PdCl₂ (0.019 g, 0.03 mmol) in EtOH:toluene=1:1 (v/v) (6 mL) was heated at 100° C. with stirring for 1.5 h. The mixture was cooled to RT, diluted with EtOAc (30 mL) and saturated brine (20 mL) and partitioned. The aqueous layer was extracted with EtOAc (3×60 mL). The combined organic extracts were dried over MgSO₄, concentrated and purified by SGC using CH₂Cl₂:MeOH as eluent (gradient from 99:1 to 98:2, v/v) to give IX-A-1 (0.121 g, quant.). ¹H NMR (400 MHz, CDCl₃) δ 1.66-1.73 (m, 2H), 1.79-1.86 (m, 2H), 2.22-2.29 (m, 2H), 2.41-2.47 (m, 2H), 4.00 (s, 3H), 6.17 (m, 1H), 7.46 (t, J 7.6, 2H), 7.55 (tt, J 7.5, 1.5, 1H), 8.00 (d, J 2.1, 1H), 8.01 (s, 1H), 8.03 (s, 1H), 8.15-8.19 (m, 2H), 8.78 (d, J 2.1, 1H).

### 4-(4-(3-(1-methyl-1H-pyrazol-4-yl)-1-(phenylsulfonyl)-1H-pyrazolo[-3,4-b]pyridin-5-yl)cyclohex-3-enyl)-1,4-oxazepane (IX-A-3)

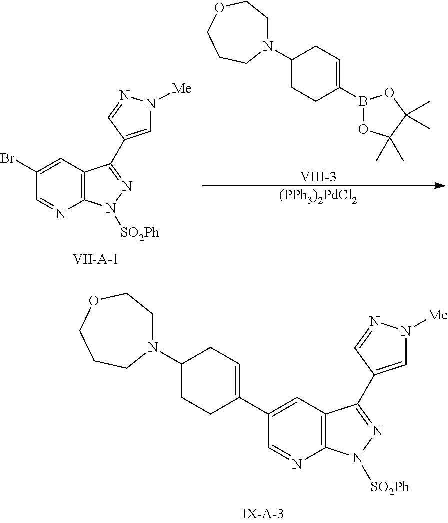

A mixture of VII-A-1 (0.115 g, 0.275 mmol), boronic ester VIII-3 (0.101 g, 0.33 mmol), LiCl (0.035 g, 0.825 mmol), 1.0 M Na₂CO₃ (0.74 mL, 0.742 mmol) and (PPh₃)₂PdCl₂ (0.019 g, 0.03 mmol) in EtOH:toluene=1:1 (v/v) (6 mL) was heated at 105° C. with stirring for 2.5 h. The mixture was cooled to RT. The mixture was diluted with brine (20 mL): EtOAc (30 mL) and partitioned. The aqueous layer was extracted with EtOAc (3×60 mL). The combined organic extracts were dried over MgSO₄, concentrated and purified by SGC using CH₂Cl₂:MeOH as eluent (gradient elution from 100:0 to 94:6, v/v) to afford the Suzuki adduct IX-A-3 (0.167 g, quant.). ¹H NMR (400 MHz, CDCl₃) δ 1.64-1.77 (m, 1H), 1.87-2.02 (m, 2H), 2.11-2.35 (m, 2H), 2.42-2.54 (m, 1H), 2.56-2.64 (m, 2H), 2.84-3.07 (m, 5H), 3.75-3.81 (m, 4H), 4.00 (s, 3H), 6.12 (m, 1H), 7.46 (t, J 7.7, 2H), 7.56 (t, J 7.6, 1H), 7.99 (d, J 2.1, 1H), 8.02 (s, 2H), 8.15-8.19 (m, 2H), 8.76 (d, J 2.1, 1H).

### 2-cyclohexenyl-7-(1-methyl-1H-pyrazol-4-yl)-5-(phenylsulfonyl)-5H-pyrrolo[3,2-b]pyrazine (IX-E-1)

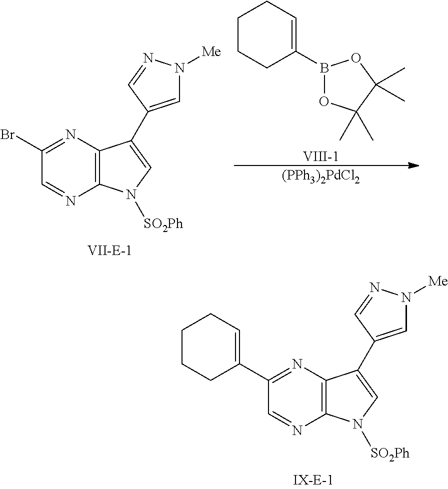

A mixture of VII-E-1 (0.33 g, 0.789 mmol), boronic ester VIII-1 (0.3284 g, 1.5779 mmol), LiCl (0.08362 g, 1.9724 mmol), 1.0 M Na₂CO₃ (7.9 mL, 7.9 mmol) and (PPh₃)₂PdCl₂ (0.05538 g, 0.0789 mmol) in EtOH:toluene=1:1 (v/v) (15.8 mL) was heated at 110° C. with stirring for 4 h. The mixture was cooled to RT. Water (40 mL) was added and the mixture was extracted with EtOAc (3×50 mL). The combined organic extracts were dried over MgSO₄, concentrated and purified by SGC using hexane:EtOAc (gradient from 9:1 to 0:10, v/v) to give IX-E-1 (0.100 g, 30%) as a yellow solid. ¹H NMR (400 MHz, CDCl₃) δ 1.68-1.77 (m, 2H), 1.80-1.89 (m, 2H), 2.31 (qd, J 6.32, 2.40, 2H), 2.62 (tq, J 6.21, 2.14, 2H), 4.00 (s, 3H), 6.74 (tt, J 3.90, 1.85, 1H), 7.46-7.53 (m, 2H), 7.59 (tt, J 6.69, 1.26, 1H), 8.01 (d, J 0.63, 1H), 8.02 (s, 1H), 8.14-8.19 (m, 3H), 8.54 (s, 1H)

### 7-(1-methyl-1H-pyrazol-4-yl)-2-[4-(morpholin-4-yl)cyclohex-1-en-1-yl]-5-(phenylsulfonyl)-5H-pyrrolo[2,3-b]pyrazine (IX-E-6)

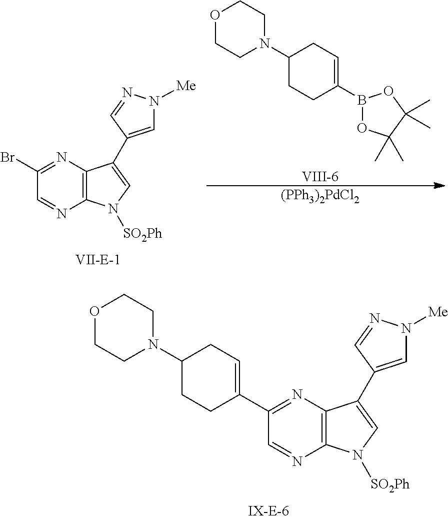

A mixture of VII-E-1 (1.66 g, 3.97 mmol), boronic ester VIII-6 (2.3273 g, 7.9374 mmol), LiCl (0.4206 g, 9.922 mmol), 1.0 M Na₂CO₃ (9.9 mL, 9.9 mmol) and (PPh₃)₂PdCl₂ (0.2786 g, 0.3969 mmol) in EtOH:toluene=1:1 (v/v) (19.8 mL) was heated at 110° C. with stirring for 2 h. The mixture was cooled to RT. Water (100 mL) was added and the mixture was extracted with EtOAc (3×100 mL). The combined organic extracts were dried over MgSO₄, concentrated and purified by SGC using hexane:EtOAc (gradient from 80:20 to 25:75, v/v) followed by CH₂Cl₂:EtOAc=1:1 (v/v) to give a pale red solid. The solid was triturated with Et₂O (30 mL) and filtered off to give IX-E-6 (1.20 g, 60%). ¹H NMR (400 MHz, CDCl₃) δ 2.14-2.41 (m, 2H), 2.46-2.80 (m, 8H), 2.88-3.01 (m, 1H), 3.78 (t, J 4.6, 4H), 4.00 (s, 3H), 6.65-6.75 (m, 1H), 7.45-7.53 (m, 2H), 7.56-7.64 (m, 1H), 8.02 (s, 1H), 8.03 (s, 1H), 8.14 (s, 1H), 8.15-8.20 (m, 2H), 8.54 (s, 1H).

### 5-bromo-3-(1-methyl-1H-pyrazol-4-yl)-1H-pyrazolo[3,4-b]pyridine (X-A-1)

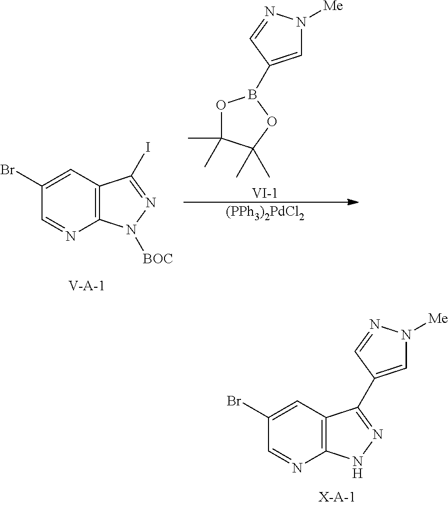

A mixture of diazaindazole V-A-1 (0.952 g, 2.24 mmol), boronic ester VI-1 (0.515 g, 2.47 mmol), LiCl (0.285 g, 6.73 mmol), 1.0 M Na₂CO₃ (6.05 mL, 6.05 mmol) and (PPh₃)₂PdCl₂ (0.157 g, 0.22 mmol) in EtOH:toluene=1:1 (v/v) (24 mL) was heated at 100° C. with stirring for 3 h. The mixture was cooled to RT, concentrated, diluted with CHCl₃ and filtered to remove the inorganics. The filtrate was concentrated. The residue was purified by SGC using CH₂Cl₂:MeOH as eluent (gradient elution from 97:3 to 90:10, v/v) to afford the Suzuki product X-A-1 as a solid (0.397 g, 64%). ¹H NMR (400 MHz, CDCl₃+trace CD₃OD) δ 4.01 (s, 3H), 7.96 (s, 1H), 8.01 (s, 1H), 8.39 (d, J 2.1, 1H), 8.59 (s, 1H).

### EXAMPLE 6

### 5-(1-methyl-1H-pyrazol-4-yl)-3-phenyl-7H-pyrrolo[2,3-e]pyridazine (11)

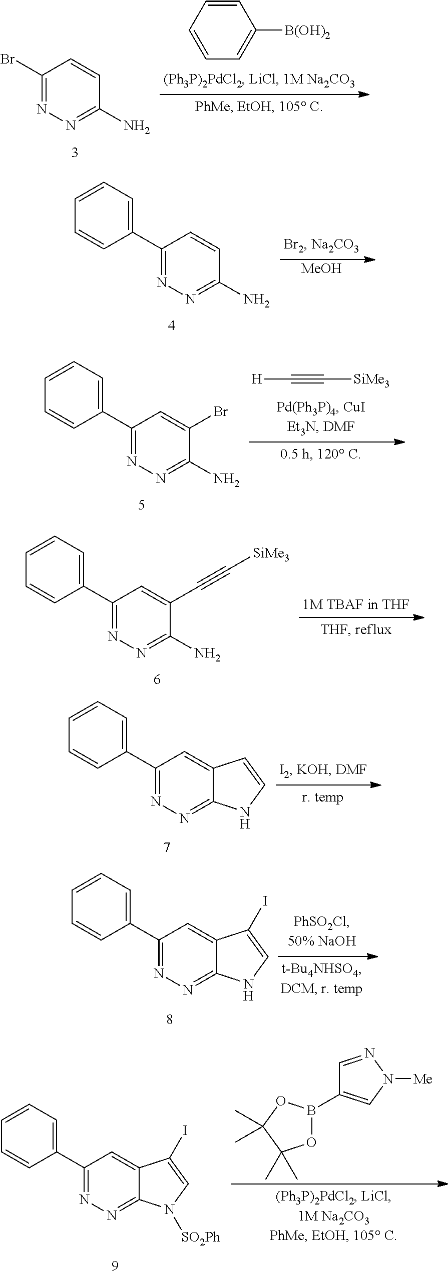

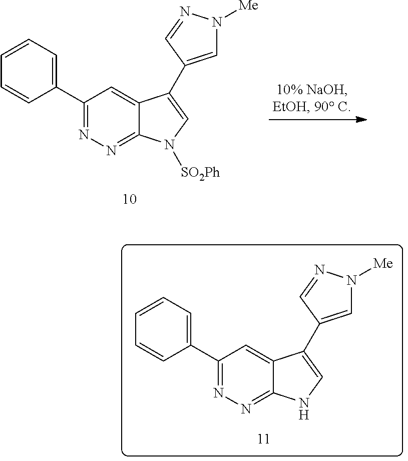

### 6-Phenylpyridazin-3-amine (4)

To a stirred solution of 6-bromo-3-pyridazinamine 3 (8.90 g, 51.1 mmol), lithium chloride (6.50 g, 153.4 mmol), dichlorobis(triphenylphosphine) palladium (II) (0.359 g, 0.511 mmol), 1 M sodium carbonate solution (138 mL, 138.1 mmol) in PhMe (130 mL) and EtOH (130 mL) was added phenylboronic acid (9.35 g, 76.7 mmol). The reaction mixture was allowed to reflux for 18.5 h and then cooled to RT and partitioned with EtOAc and saturated brine. The aqueous layer was washed with EtOAc (3×). The combined organic extracts were dried (MgSO₄), filtered and evaporated to afford a white crystalline solid 4 (7.84 g, 89%). ¹H NMR (400 MHz; CDCl₃) δ 4.75 (brs, 2H), 6.84 (d, J=9.1 Hz, 1H), 7.41 (tt, J=7.5 and 1.5 Hz, 1H), 7.45-7.51 (m, 2H), 7.65 (d, J=9.1 Hz, 1H) and 7.94-7.98 (m, 2H).

### 4-Bromo-6-phenylpyridazin-3-amine (5)

To a stirred solution of the pyridazine 4 (7.84 g, 45.8 mmol) and sodium hydrogen carbonate (11.5 g, 137.4 mmol) in MeOH (400 mL) was added a solution of bromine (3.53 mL, 68.7 mmol) in MeOH (30 mL) dropwise over 11 min. After a further 2 h, the mixture was vacuum filtered through a sinter funnel and the solid residue washed with MeOH. The combined filtrates were evaporated and the resulting residue re-dissolved in EtOAc and washed with saturated sodium thiosulfate solution (1×) and saturated brine (1×). The organic extract was dried (MgSO₄), filtered and evaporated. The crude material was purified by flash silica chromatography using gradient elution (hexane to 3/1 to 2/1 to 1/1 hexane, EtOAc) to afford the bromide 5 (5.431 g, 47.4%). ¹H NMR (400 MHz; CDCl₃) δ 5.22 (brs, 2H), 7.43 (tt, J 7.4 and 1.4 Hz, 1H), 7.45-7.51 (m, 2H), 7.91 (s, 1H) and 7.92-7.97 (m, 2H). MS (ES) m/z 250 (M⁺).

### 6-Phenyl-4-[(trimethylsilyl)ethynyl]pyridazin-3-amine (6)

A solution of the bromide 5 (3 g, 12 mmol), copper (I) iodide (0.274 g, 1.4 mmol), tetrakis(triphenylphosphine)palladium(0) (0.692 g, 0.6 mmol), ethynyltrimethylsilane (1.99 mL, 14.4 mmol), triethylamine (18 mL, 129.4 mmol) in DMF (60 mL) was stirred at 120° C. After 0.5 h the mixture was allowed to cool to RT and then evaporated on a cold-finger. The crude residue was purified by flash silica chromatography using gradient elution (hexane to 1/1 hexane, EtOAc) to afford the silane 6 (2.104 g, 65.6%) as a tan solid. ¹H NMR (400 MHz; CDCl₃) δ 0.31 (s, 9H), 5.27 (brs, 2H), 7.41 (tt, 7.3 and 1.4 Hz, 1H), 7.44-7.49 (m, 2H), 7.67 (s, 1H) and 7.92-7.96 (m, 2H). MS (ES) m/z 268 (MH⁺).

### 3-Phenyl-7H-pyrrolo[2,3-c]pyridazine (7)

To a solution of the silane 6 (58.9 mg, 0.22 mmol) in THF (2.5 mL) was added dropwise a solution of 1 M TBAF in THF (0.48 mL, 0.48 mmol) over 1.5 min. The mixture was allowed to reflux for 6.5 h and then cooled and evaporated. The residue was diluted with CH₂Cl₂ and water and partitioned. The aqueous layer was washed with CH₂Cl₂ (3×). The combined organic extracts were dried (MgSO₄), filtered and evaporated. The crude material was purified on a 1×1 mm PTLC plate using EtOAc as eluent to afford the 6,7-diazaindole 7 (34.9 mg, 81%). ¹H NMR (400 MHz; CDCl₃) δ 6.61 (d, J=3.6 Hz, 1H), 7.46 (tt, J 7.4 and 1.4 Hz, 1H), 7.51-7.57 (m, 2H), 7.71 (d, 3.6 Hz, 1H), 8.07-8.10 (m, 2H), 8.11 (s, 1H) and 10.58 (brs, NH). MS (ES) m/z 196 (MH⁺).

### 5-Iodo-3-phenyl-7H-pyrrolo[2,3-c]pyridazine (8)

To a stirred solution of the pyrrolo-pyradazine 7 (348.4 mg, 1.79 mmol) in DMF (15 mL) was added crushed KOH (378 mg, 6.74 mmol) in one portion. After 10 min, iodine (408 mg, 1.61 mmol) was added in one portion and stirred for a further 1.5 h. The mixture was partially evaporated and then diluted with EtOAc and saturated sodium hydrogen carbonate solution and partitioned. The aqueous layer was washed with EtOAc (4×) and the combined organic extracts dried (MgSO₄), filtered and evaporated to afford the iodide 8 (1.743 g) which was used directly without any purification. MS (ES) m/z 322 (MH⁺).

### 5-Iodo-3-phenyl-7-(phenylsulfonyl)-7H-pyrrolo[2,3-c]pyridazine (9)

To a stirred solution of the iodide 8 (assumed theoretical yield 0.574 g, 1.79 mmol), tetrabutylammonium hydrogensulfate (0.091 g, 0.27 mmol) in 50% NaOH (1 mL) and CH₂Cl₂ (20 mL) was added benzenesulfonyl chloride (0.35 mL, 2.77 mmol). After 70 min the mixture was diluted with CH₂Cl₂ and saturated brine and partitioned. The aqueous layer was washed with CH₂Cl₂ (4×) and the combined organic extracts dried (MgSO₄), filtered and evaporated to afford a solid. This was diluted with cold MeOH and stirred at RT for 1 h. The resulting solid was vacuum filtered through a Kiriyama funnel to afford the phenylsulfonylated pyrrolo-pyridazine 9 as a powder (0.544 g, 66%, 2 steps). ¹H NMR (400 MHz; CDCl₃) δ 7.46-7.58 (m, 5H), 7.65 (tt, J=7.4 and 1.4 Hz, 1H), 7.77 (s, 1H), 8.07-8.10 (m, 3H) and 8.36-8.40 (m, 2H). MS (ES) m/z 462 (MH⁺).

### 5-(1-Methyl-1H-pyrazol-4-yl)-3-phenyl-7-(phenylsulfonyl)-7H-pyrrolo[2,3-c]pyridazine (10)

To a solution of the protected pyrrolo-pyridazine 9 (100 mg, 0.217 mmol) in PhMe (4 mL) and EtOH (4 mL) was added lithium chloride (27.5 mg, 0.65 mmol), dichlorobis(triphenylphosphine) palladium (II) (1.5 mg, 0.002 mmol), 1-methyl-4-(4,4,5,5-tetramethyl-1,3,2-dioxaborolan-2-yl)-1H-pyrazole (58.6 mg, 0.282 mmol) and 1 M sodium carbonate (0.58 mL, 0.58 mmol). After 2.5 h at reflux, the mixture was diluted with EtOAc and saturated sodium hydrogen carbonate solution and partitioned. The aqueous layer was washed with EtOAc (3×). The combined organic extracts were dried (MgSO₄), filtered and evaporated. This material was purified on 2×1 mm PTLC plates using 5/1 DCM, EtOAc as eluent to afford the Suzuki adduct 10 as a cream powder (81.3 mg, 90%). ¹H NMR (400 MHz; CDCl₃) δ 4.02 (s, 3H), 7.45-7.57 (m, 5H), 7.63 (tt, J=7.4 and 1.4 Hz, 1H), 7.70 (s, 1H), 7.80 (d, J=0.8 Hz, 1H), 7.99 (s, 1H), 8.04 (s, 1H), 8.04-8.08 (m, 2H) and 8.36-8.42 (m, 2H). MS (ES) m/z 416 (MH⁺).

### 5-(1-Methyl-1H-pyrazol-4-yl)-3-phenyl-7H-pyrrolo[2,3-c]pyridazine (11)

To a solution of the pyrrolo-pyridazine 10 (81.3 mg, 0.196 mmol) in ethanol (10 mL) was added a 10% NaOH solution (0.8 mL) and the mixture heated at 90° C. After 40 minutes the mixture was cooled to RT and diluted with EtOAc (20 mL). The mixture was washed with saturated NaHCO₃ solution (3×15 mL) and saturated brine (1×15 mL). The organic extracts were dried (MgSO₄), filtered and evaporated. The resulting residue was purified on a 1×1 mm PTLC plate using EtOAc as eluent to afford the deprotected pyrrolo-pyridazine 11 (21.6 mg, 40%). ¹H NMR (400 MHz; CDCl₃) δ 4.02 (s, 3H), 7.48 (t, J=7.4 Hz, 1H), 7.56 (t, J=7.4 Hz, 2H), 7.68 (s, 1H), 7.81 (d, J=1.3 Hz, 2H), 8.10 (d, J=7.4 Hz, 2H), 8.18 (s, 1H) and 11.44 (brs, NH). MS (ES) m/z 276 (MH⁺).

### Biological Activity

### JNK1, JNK2, JNK3—SPA Assay

1. The compound was dissolved in DMSO to a convenient concentration and this was diluted in 10% DMSO to a five times concentrate of the desired starting concentration (frequently 1:100).
2. 10 μl of 500 mM EDTA was added to alternative wells of the Opti-plate row, which would receive kinase reaction plus DMSO. This created the negative control.
3. For the JNK2 and JNK3 assay, compounds were prepared in six 2-fold dilutions with water and each concentration is tested in duplicate. For the JNK1 assay compounds are prepared in four 5-fold dilutions with water which are tested in triplicate. Controls were treated identically.
4. 20 μA per well of each compound concentration was transferred to an Opti-plate, in duplicate.
5. 30 μl (JNK2/3 SPA) or 50 μl (JNK1 SPA) of substrate solution (25 mM HEPES pH 7.5, 10 mM magnesium acetate with 3.33 μM ATP (JNK2/3) or 2 μM ATP (JNK1), approximately 7.5 kBq [γ-³³P] ATP, GST-c-Jun, in water) was added to each well.
6. 50 μl (JNK2/3 SPA) or 30 μl (JNK1 SPA) of kinase solution (JNK in 25 mM HEPES pH 7.5, 10 mM Mg Acetate) was added to each well.

| Kinase | Kinase per well (μg) | GST-c-Jun per well (μg) |
|---|---|---|
| JNK1 | 0.25 | 1 |
| JNK2 | 0.2 | 1.2 |
| JNK3 | 0.16 | 1.2 |

7. The plate was incubated for 30 minutes at room temperature.
8. 100 μl of bead/stop solution was added to each well (5 mg/ml glutathione-PVT-SPA beads, 40 mM ATP in PBS).
9. Plates were sealed and incubated for 30 minutes at room temperature, centrifuged for 10 minutes at 2500 g and counted.
10. The IC₅₀ values were calculated as the concentration of the compound being tested at which the phosphorylation of c-Jun was decreased to 50% of the control value. IC₅₀ values for example compounds of this invention are given in Table 1.

TABLE 1
IC₅₀ values for compounds (I) against JNK3

| Example no. | IC₅₀ [nM] |
|---|---|
| 1 (I-A-2) | 1700 |
| 2 (I-A-4) | >2000 |
| 3 (I-A-5) | >2000 |
| 4 (I-E-2) | 178 |

1. A compound of formula (I) or a pharmaceutically acceptable salt thereof: 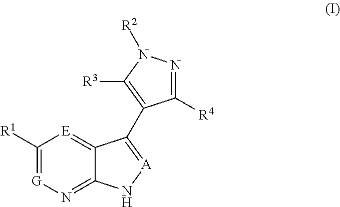
wherein
A is CH or N;
E is CH or N;
G is CH or N;
A is N when E and G are CH;
E is N when A and G are CH;
G is N when A and E are CH;
R¹ is a 5-7 membered non-aromatic hydrocarbon cyclic group optionally and independently substituted with 1-4 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵, NR⁵R⁶ and —R^{a}—R^{b};
or R¹ is a 6-10 membered aromatic or partially saturated hydrocarbon cyclic group, optionally and independently substituted with 1-6 substituent(s) selected from the group consisting of halogen, cyano, hydroxy, oxo, ethylenedioxy, C₁₋₆alkyl, C₁₋₆alkoxy, C₁₋₆hydroxyalkyl, —C(O)OH, —CONH₂, NHR⁵ and NR⁵R⁶;
R^{a} is a single bond or —CH₂—;
R^{b} is a 4-8 membered non-aromatic heterocyclic group, C₆₋₁₀aryl or a 5-7 membered heteroaryl group, optionally and independently substituted with 1-4 substituent(s) selected from the group consisting of halogen and C₁₋₆alkyl;
R⁵ and R⁶ are independently selected from C₁₋₆alkyl, C₁₋₆alkoxy, C₁₋₆hydroxyalkyl or a 6-membered non-aromatic heterocyclic group;
and two or more positions on R¹ are optionally bridged by a group —X— wherein X is O, CH₂, CH₂—CH₂, NR⁷, CH₂—CH₂—CH₂, CH₂—CH(CH₂—)—CH₂ or N(R⁷)—CH(CH₂—)CH₂ to form a bicyclic or tricyclic ring system, wherein R⁷ is independently selected from hydrogen or C₁₋₆alkyl and wherein said bridge may be optionally and independently substituted with one or more of C₁₋₆alkyl, cyano, CO₂NH₂, C₁₋₆hydroxyalkyl, oxo, hydroxy, C₁₋₆ alkylamino or a 6-membered non-aromatic heterocyclic group;
R² is hydrogen, C₁₋₆alkyl optionally substituted with a 4-7 membered non-aromatic heterocyclic group, or C₁₋₆haloalkyl;
R³ is hydrogen or C₁₋₆alkyl; and
R⁴ is hydrogen or C₁₋₆alkyl.

2. The compound or a pharmaceutically acceptable salt as claimed in claim 1 wherein R¹ is a 5-7 membered non-aromatic hydrocarbon cyclic group optionally and independently substituted with 1-3 substituent(s) selected from the group consisting of halogen, oxo, ethylenedioxy, C₁₋₆alkyl, C₁₋₆alkoxy, C₁₋₆hydroxyalkyl, —C(O)OH and —R^{a}—R^{b};
R^{a} is a single bond or —CH₂—; and
R^{b} is a 4-7 membered non-aromatic heterocyclic group, C₆₋₁₀aryl or a 5-6 membered heteroaryl group, optionally and independently substituted with 1-3 substituent(s) selected from the group consisting of halogen and C₁₋₆alkyl.

3. The compound or a pharmaceutically acceptable salt thereof as claimed in claim 1 wherein R² is methyl, morpholinoethyl or trifluoromethyl.

4. The compound or a pharmaceutically acceptable salt thereof as claimed in claim 1 wherein R³ is hydrogen or methyl.

5. The compound or a pharmaceutically acceptable salt thereof as claimed in claim 1 wherein R⁴ is hydrogen or methyl.

6. The compound or a pharmaceutically acceptable salt thereof as claimed in claim 1 of formula (Ia): 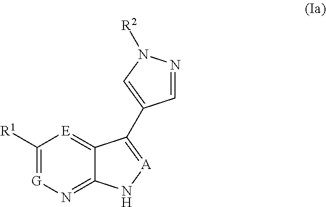
where A, E, G, R¹ and R² are as defined in claim 1.

7. The compound or a pharmaceutically acceptable salt thereof as claimed in claim 1 of formula (Ib): 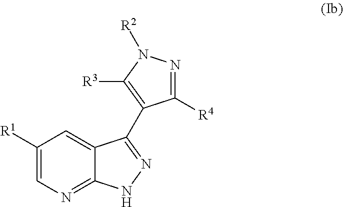
where R¹, R², R³ and R⁴ are as defined in claim 1.

8. The compound or a pharmaceutically acceptable salt thereof as claimed in claim 1 of formula (Ic): 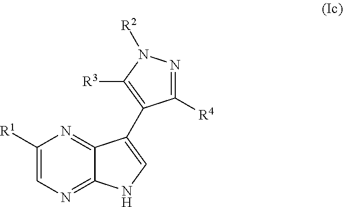
where R¹, R², R³ and R⁴ are as defined in claim 1.

9. The compound or a pharmaceutically acceptable salt thereof as claimed in claim 1 of formula (Id): 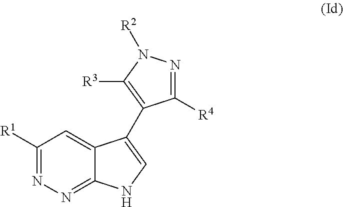
where R¹, R², R³ and R⁴ are as defined in claim 1.

10. The compound as claimed in claim 1 selected from the following group or a pharmaceutically acceptable salt thereof:
5-cyclohexyl-3-(1-methyl-1H-pyrazol-4-yl)-1H-pyrazolo[3,4-b]pyridine,
4-((1r,4s)-4-(3-(1-methyl-1H-pyrazol-4-yl)-1H-pyrazolo[3,4-b]pyridin-5-yl)cyclohexyl)-1,4-oxazepane,
4-((1s,4s)-4-(3-(1-methyl-1H-pyrazol-4-yl)-1H-pyrazolo[3,4-b]pyridin-5-yl)cyclohexyl)-1,4-oxazepane,
2-cyclohexyl-7-(1-methyl-1H-pyrazol-4-yl)-5H-pyrrolo[3,2-b]pyrazine,
4-((1r,4r)-4-(7-(1-methyl-1H-pyrazol-4-yl)-5H-pyrrolo[3,2-b]pyrazin-2-yl)cyclohexyl)morpholine, and
5-(1-methyl-1H-pyrazol-4-yl)-3-phenyl-7H-pyrrolo[2,3-c]pyridazine.

11. A pharmaceutical composition comprising the compound according to claim 1, or a pharmaceutically acceptable salt thereof, in association with a pharmaceutically acceptable carrier.

12. The compound according to claim 1 for use in medicine.

13. The compound according to claim 1 for preventing or treating a neurodegenerative disorder, an inflammatory disease, an autoimmune disease or organ failure.

14. The compound according to claim 13, wherein the neurodegenerative disorder is multiple sclerosis.

15. The compound according to claim 13, wherein the autoimmune disease is rheumatoid arthritis.

16. The compound according to claim 1 for preventing or treating diabetic nephropathy, heart failure or liver failure.

17. A method for preventing or treating a neurodegenerative disorder, an inflammatory disease, an autoimmune disease or organ failure, which comprises administering to a mammalian animal an effective amount of the compound or pharmaceutically acceptable salt thereof according to claim 1.

18. Use of the compound or pharmaceutically acceptable salt thereof, according to claim 1, for the manufacture of a medicament for the prevention or treatment of a neurodegenerative disorder, an inflammatory disease, an autoimmune disease or organ failure.
